SEQUENCE LISTING (2 sequences)

SEQ ID NO: 1 - DNA, 1833 bp, Drosophila melanogaster
  CDS (1)..(1833)
atgatcaatatcgctggcgtggtgagcatcgtgctcttctacctcctgatcctggtcgttggcatttgggccggtcgcaagaagcagtccggcaatgattcggaggaggaggtcatgctggccggacgctccatcggcctcttcgtgggcatcttcaccatgacggccacctgggtgggtggcggctacatcaacggcacggcggaggctatatacacatcgggtctggtgtggtgccaggctccatttggatacgctctaagcttggtatttggtggcatcttctttgccaatcccatgcgcaagcagggttacatcaccatgttggatccgttgcaggattcctttggtgagcggatgggaggattgctcttcctgcccgctctatgcggtgaggtcttttgggcagccggcatcctggctgcacttggcgccactctatcggtgatcatcgacatggatcaccgcacctcggtgatcctgtcctcctgcatcgccatcttctacacactgttcggtggactgtactccgtggcgtatacggacgtgatccagttgttctgcatcttcatcggtctgtggatgtgcattcccttcgcctggagcaacgagcacgtgggcagcctgagtgacctggaggtggattggattgggcacgtggagcctaaaaagcattggctgtacatagactacggcttgctgctcgtctttggtggcattccctggcaggtctacttccagcggcaaaacggcaggaagggcccagcttctgcctatgttgcagccgccggatgcattttgatggccattcccccggtgctcatcggagcgattgccaaggctacaccttggaacgagacagattacaagggaccctatcccctgaccgtggacgagacgagcatgattctgcccatggtgctgcagtacctcacgcctgacttcgtgtccttctttggattgggcgctgtttccgccgccgtgatgtcctccgccgactcctcggtgctctccgccgcctccatgttcgctcggaacgtgtacaaattgattttccgtcagaaggcgtccgagatggaaatcatttgggtgatgcgagtcgccatcattgtggtgggcatcctggctaccatcatggccctcaccattccctccatctacggtttgtggtccatgtgctcggatctggtctacgtcattctgttcccgcagctactgatggtggtgcacttcaagaagcactgcaacacgtacggcagcctgtcggcatacattgtggccctggccatccgactgtcgggcggtgaggccatcttgggactggctccattgatcaagtatcccggctacgacgaggagaccaaggagcagatgttccccttccgcaccatggccatgctgctcagcctggtcacgctcatctcggtctcctggtggactaaaatgatgtttgagtccggcaagttgccgcccagctacgactacttccgctgtgtggtcaatattccggaggatgtgcagcgtgtgggcgatccctcggagtcgggtgagcagctatccgtgatggctggaccgctggcccgatcctacggagcggccaccatggcgggcaaggatgagcgcaatggccgcatcaatcccgccctggaatcggacgacgatctgccggtggcggaggcacgtcgcatcaaccaggagacggcgcaggcgcaggtcaaaaagatgctggataacgccactggggtgaagccgtcgggcggaggcggtggtcacctccagagccaaagcgggatggccatgcccacggcggagcaggacaatacggccttctga

SEQ ID NO: 2 - protein, 610 aa, Drosophila melanogaster
MINIAGVVSIVLFYLLILVVGIWAGRKKQSGNDSEEEVMLAGRSIGLFVGIFTMTATWVGGGYINGTAEAIYTSGLVWCQAPFGYALSLVFGGIFFANPMRKQGYITMLDPLQDSFGERMGGLLFLPALCGEVFWAAGILAALGATLSVIIDMDHRTSVILSSCIAIFYTLFGGLYSVAYTDVIQLFCIFIGLWMCIPFAWSNEHVGSLSDLEVDWIGHVEPKKHWLYIDYGLLLVFGGIPWQVYFQRQNGRKGPASAYVAAAGCILMAIPPVLIGAIAKATPWNETDYKGPYPLTVDETSMILPMVLQYLTPDFVSFFGLGAVSAAVMSSADSSVLSAASMFARNVYKLIFRQKASEMEIIWVMRVAIIVVGILATIMALTIPSIYGLWSMCSDLVYVILFPQLLMVVHFKKHCNTYGSLSAYIVALAIRLSGGEAILGLAPLIKYPGYDEETKEQMFPFRTMAMLLSLVTLISVSWWTKMMFESGKLPPSYDYFRCVVNIPEDVQRVGDPSESGEQLSVMAGPLARSYGAATMAGKDERNGRINPALESDDDLPVAEARRINQETAQAQVKKMLDNATGVKPSGGGGGHLQSQSGMAMPTAEQDNTAF

(19) United States
(12) Patent Application Publication
(10) Pub. No.: US 2004/0048261 A1
(43) Pub. Date: Mar. 11, 2004

(54) Invertebrate choline transporter nucleic acids, polypeptides and uses thereof

(76) Inventor: Klaus Raming, Leverkusen (DE)

Correspondence Address: BAYER CROPSCIENCE LP, Patent Department, 100 BAYER ROAD, PITTSBURGH, PA, 15205-9741, US

(21) Appl. No.: 10/241,784

(22) Filed: Sep. 11, 2002

(51) Int. Cl. C12Q 1/68; A01K 67/033; C07H 21/04; C07K 14/705

(52) U.S. Cl. 435/6; 435/69.1; 435/320.1; 435/348; 530/350; 536/23.5; 800/8

(57) ABSTRACT

Nucleic acid encoding a choline transporter from Drosophila melanogaster, herein referred to as dmCHT, is provided. The dmCHT nucleic acids, fragments and derivatives thereof may be used to genetically alter animals, such as insects, arachnids, worms, or in cultured cells to result in dmCHT expression or mis-expression. The thus genetically altered animal or cell may find use in various screening assays to identify potential innovative pesticides or therapeutic agents by virtue of interaction with the dmCHT. The genetically altered organism or cell may also find use in studying dmCHT activity and identifying other genes that modulate the function of, or interact with, the dmCHT gene.

### FIELD OF THE INVENTION

The present invention relates, in general, to nucleic acids and polypeptides, and more specifically, to nucleic acid encoding an invertebrate choline transporter and the uses thereof in identifying potential pesticides.

### BACKGROUND OF THE INVENTION

Synaptosome studies in insects and mammals have demonstrated two carrier-mediated transport systems for choline uptake. At high concentrations, choline is transported primarily by a low-affinity, sodium-independent system that is inhibited by hemicholinium-3 (HC-3) with a high inhibition constant (Kᵢ) of approximately 50 μM. The low-affinity system is thought to be ubiquitous in cells and required for phosphatidylcholine synthesis. At low concentrations, choline is transported by a high-affinity, sodium-dependent system that is inhibited by HC-3 with a low Kᵢ of 10-100 nM. The high-affinity system is postulated to be present specifically in cholinergic neurons, because a substantial proportion of choline is converted to acetylcholine only if taken up through the high-affinity system.

The theory that the high-affinity choline transport system is unique to cholinergic neurons is further supported in a variety of denervation studies by the selective loss of the high-affinity choline uptake following depletion of cholinergic terminals. Choline uptake is generally believed to be the rate-limiting step in acetylcholine synthesis. The high-affinity choline transporter has been cloned and expressed from C. elegans (Okuda T, et al., Nature Neurosci. 2000 3:120-125), rat (Okuda T, et al., supra) and human (Apparsundaram S, et al., Biochem Biophys Res Commun 2000 276:862-867).

Pesticide development has traditionally focused on the chemical and physical properties of the pesticide itself, thereby making it a relatively time-consuming and expensive process. Consequently, efforts heretofore have concentrated on the modification of pre-existing and well-validated compounds, rather than on the development of innovative pesticides. A promising alternative, therefore, may be to identify and validate biological targets against which potential ligands may be screened (Margolis and Duyk, Nature Biotech. (1998) 16:311). The development of new compounds that are safer, more selective and more efficient may be hastened through such target-based discovery approaches.

The essential functions of target genes in insects and nematodes may be tested directly by powerful genetic methods, thereby eliminating the costly uncertainty of whether a specific gene or biochemical activity might be a pesticide target. Because the phenotypic consequence of genetically modulated target gene activity may act as a surrogate for chemical inhibition or activation of a protein target, genes that kill the organism if over-expressed or knocked out represent first-stage validated targets.

High-throughput screening assays may be employed in testing a compound for its ability to interfere in vitro with the normal activity of the target, thereby identifying compounds that have the same effect on the organism. Biological definition of targets provides the opportunity to optimize the chemistry around validated targets. Upon identification, the molecular diversity inherent in the structures of the targets may be exploited via combinatorial chemistry and high-throughput screening.

High-throughput assays may provide access to the structural variety granted by combinatorial chemistry and have the dual advantages of speed and low cost. A further advantage is that potential lead compounds may be directly counter-screened on the same target, cloned from human or beneficial insect sources, to exclude broad-spectrum toxins.

### SUMMARY OF THE INVENTION

Accordingly, the present invention provides invertebrate homologs of a Drosophila melanogaster choline transporter, hereinafter referred to as dmCHT, that may be employed in genetic screening methods to characterize pathways in which the dmCHT may be involved as well as other interacting genetic pathways.

The present invention also provides methods for screening compounds that interact with the dmCHT, such as those that may find use as therapeutics or pesticides.

The advantages and benefits of the present invention will be apparent from the Detailed Description of the Invention herein below.

### BRIEF DESCRIPTION OF THE FIGURES

The present invention will now be described for purposes of illustration and not limitation in conjunction with the figures, wherein:

FIG. 1 provides the nucleotide sequence of a cDNA molecule sequence that encodes the Drosophila melanogaster choline transporter protein of the present invention (SEQ ID NO:1); and

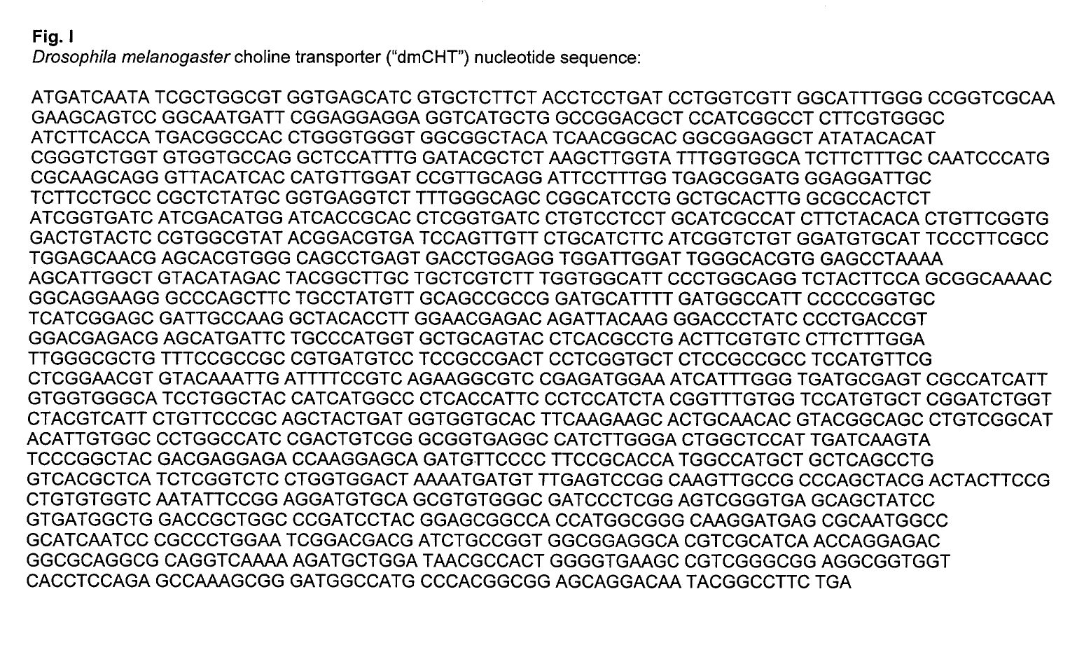

FIG. 2 provides the predicted amino acid sequence of the Drosophila melanogaster choline transporter of the present invention (SEQ ID NO:2).

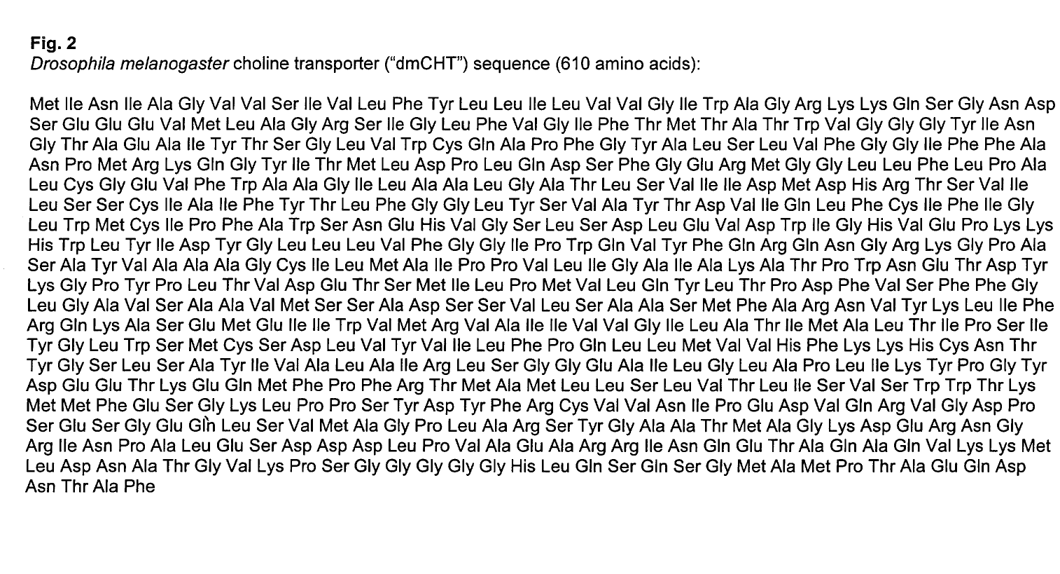

### DETAILED DESCRIPTION OF THE INVENTION

The present invention will now be described for purposes of illustration and not limitation. The contents of all references, including the patent applications cited herein, are incorporated by reference in their entireties.

The present invention describes the identification and characterization of Drosophila melanogaster (hereinafter referred to as “Drosophila”) choline transporter, hereinafter referred to as “dmCHT”. Isolated nucleic acid molecules are described herein which contain nucleotide sequences encoding the dmCHT polypeptides as well as fragments and derivatives thereof. Methods of using the isolated nucleic acid molecules and fragments of the present invention as biopesticides are also described herein, such as use of RNA interference methods to block dmCHT activity. Vectors and host cells containing the dmCHT nucleic acid of the present invention are provided, as well as metazoan invertebrate organisms (e.g. insects, coelomates and pseudocoelomates) genetically modified to express or mis-express the dmCHT polypeptides of the present invention.

An important application of the dmCHT nucleic acids, polypeptides and fragments thereof of the present invention lies in screening assays to identify compounds having potential as pesticides or therapeutic agents, that interact with dmCHT proteins. Such assays may involve exposing and/or contacting the dmCHT polypeptides, fragments or derivatives thereof of the present invention to one or more compounds and detecting any interaction between the compound and the dmCHT. The assays may involve administering the compound(s) to cultures of cells genetically engineered to express the dmCHT polypeptides, or alternatively, exposing, feeding, injecting, or contacting the compound(s) to an organism genetically modified to express the dmCHT polypeptides, fragments and derivatives thereof of the present invention.

Due to its ability to rapidly carry out large-scale, systematic genetic screens, an invertebrate model organism, such as Drosophila, is preferred for analyzing the expression and mis-expression of the dmCHT polypeptides, fragments or derivatives thereof of the present invention. Therefore, the present invention may provide a superior approach for identifying other components involved in the synthesis, activity and regulation of dmCHT proteins.

Systematic genetic analysis of dmCHTs using invertebrate model organisms may lead to the identification and validation of pesticide targets directed to components of the dmCHT pathway. Model organisms or cultured cells genetically altered to express the dmCHT polypeptides, fragments or derivatives thereof of the present invention may be used to screen for a compound's ability to modulate dmCHT expression or activity. Such organisms and/or cells may also be employed in the identification of new drug targets, therapeutic agents, diagnostics and prognostics for the treatment of disorders associated with ion channels. Further, such invertebrate model organisms may screen and/or identify pesticide targets directed to components of the dmCHT pathway.

Genetically altered animals may preferably be exploited in methods for studying dmCHT activity. Such methods may involve detecting the phenotype caused by the expression or mis-expression of the dmCHT polypeptides of the present invention. The methods of the present invention may additionally include detecting the phenotype of a second animal that has the same genetic modification as the first animal and a mutation in a gene of interest. Any difference between the phenotypes of the two animals identifies the gene of interest as capable of modifying the function of the gene encoding the dmCHT protein.

Information on the Sequence Listing

SEQ ID NO: 1 gives the nucleotide sequence of the isolated Drosophila melanogaster choline transporter (“dmCHT”) nucleic acid of the present invention. SEQ ID NO: 2 gives the amino acid sequence of the polypeptide deduced from the dmCHT nucleotide sequence of SEQ ID NO: 1.

I. Drosophila melanogaster Choline Transporter (dmCHT) Nucleic Acids

The present invention provides an isolated nucleic acid of a choline transporter of Drosophila, having the nucleotide sequence given by SEQ ID NO:1, as well as fragments and derivatives thereof, as described in detail herein below and the reverse complements thereof. The nucleic acid, fragments and derivatives thereof of the present invention include RNA molecules containing the nucleotide sequence of SEQ ID NO:1, fragments or derivatives thereof wherein the base uracil (U) is substituted for the base thymine (T). The DNA and RNA sequences of the present invention may be single- or double-stranded. Therefore, the term “isolated nucleic acid” herein includes, unless otherwise indicated, the reverse complement, RNA equivalent, DNA or RNA single- or double-stranded sequences and DNA/RNA hybrids of the sequence being described.

Fragments of the dmCHT nucleic acid of the present invention may be used for a variety of purposes. Interfering RNA (RNAi) fragments, particularly double-stranded (ds) RNAi, may be employed in generating loss-of-function phenotypes, or in formulating biopesticides as discussed herein below. The dmCHT nucleic acid fragments of the present invention may also function as nucleic acid hybridization probes and replication/amplification primers. Certain “antisense” fragments, i.e. reverse complements of portions of the coding sequence of SEQ ID NO:1, may inhibit the function of dmCHT proteins. The fragments may preferably be of a length sufficient to specifically hybridize with the corresponding SEQ ID NO:1. The nucleic acid fragments of the present invention may contain at least 12, preferably at least 24, more preferably at least 36 and most preferably at least 96 contiguous nucleotides of SEQ ID NO:1. Where the fragments of the present invention are flanked by other nucleotide sequences, the total length of the combined nucleic acid may be less than about 15 kb, preferably less than about 10 kb, more preferably less than about 5 kb, still more preferably less than about 2 kb and may, in some instances, be less than about 500 bases.

In one embodiment of the present invention, the nucleic acid may contain only SEQ ID NO:1 or fragments thereof. In other embodiments, the nucleic acid and fragments thereof may be joined to other components including, but not limited to, labels, peptides, agents facilitating transport across cell membranes, hybridization-triggered cleavage agents and intercalating agents.

The nucleic acids, fragments and derivatives thereof of the present invention may comprise part of a larger nucleotide sequence by being joined to other nucleic acids. The nucleic acids, fragments and derivatives thereof of the present invention may be of synthetic (non-natural) origin and/or may be isolated and/or may be purified, i.e. unaccompanied by at least some of the material with which it is associated in its natural state. Preferably, the isolated nucleic acids of the present invention constitute at least about 0.5% and more preferably at least about 5% by weight, of the total nucleic acid present in a given fraction and may preferably be recombinant. By “recombinant”, the inventor herein means containing a non-natural sequence or a natural sequence joined to nucleotide(s) other than that to which it is joined on a natural chromosome.

Derivative nucleic acids of the dmCHT nucleic acid of the present invention include those that hybridize to the nucleic acid of SEQ ID NO:1 under stringency conditions such that the hybridizing derivative nucleic acid is related to the subject nucleic acid by a certain degree of sequence identity. A nucleic acid molecule is said to be “hybridizable” to another nucleic acid molecule, such as a cDNA, genomic DNA, or RNA, where a single-stranded form of the nucleic acid molecule can anneal to the other nucleic acid molecule. “Stringency of hybridization” refers to those conditions under which nucleic acids are hybridizable. As those skilled in the art are aware, the degree of stringency may be controlled by temperature, ionic strength, pH and the presence of denaturing agents such as formamide during hybridization and washing.

The term “stringent hybridization conditions” herein means those conditions normally used by one skilled in the art to establish at least a 90% sequence identity between complementary pieces of DNA or DNA and RNA. “Moderately stringent hybridization conditions” may be used to find derivatives having at least 70% sequence identity. Finally, “low-stringency hybridization conditions” may be used to isolate derivative nucleic acid molecules sharing at least about 50% sequence identity with the nucleotide sequence of the present invention.

The ultimate hybridization stringency reflects both the actual hybridization conditions, as well as the washing conditions following the hybridization. It is well known in the art how to vary those conditions to obtain the desired result. Conditions routinely employed are set out in available procedure texts, such as Current Protocol in Molecular Biology, Vol. 1, Chap. 2.10, John Wiley & Sons, Publishers (1994) and Sambrook et al., Molecular Cloning, Cold Spring Harbor (1989).

Some preferred derivative nucleic acids of the present invention are those capable of hybridizing to SEQ ID NO:1 under stringent hybridization conditions including: prehybridization of filters containing nucleic acid for 8 hours to overnight at 65° C. in a solution containing 6× single strength citrate (SSC) (1× SSC is 0.15 M NaCl, 0.015 M Na citrate; pH 7.0), 5× Denhardt's solution, 0.05% sodium pyrophosphate and 100 μg/ml herring sperm DNA; hybridization for 18-20 hours at 65° C. in a solution containing 6× SSC, 1× Denhardt's solution, 100 μg/ml yeast tRNA and 0.05% sodium pyrophosphate; and washing of filters at 65° C. for 1 hour in a solution containing 0.2× SSC and 0.1% SDS (sodium dodecyl sulfate).

Other preferred derivative nucleotide sequences of the present invention having at least about 70% sequence identity with SEQ ID NO:1 are those capable of hybridizing to SEQ ID NO:1 under moderately stringent conditions including: pretreatment of filters containing nucleic acid for 6 hours at 40° C. in a solution containing 35% formamide, 5× SSC, 50 mM Tris-HCl (pH7.5), 5 mM EDTA, 0.1% PVP, 0.1% Ficoll, 1% BSA and 500 μg/ml denatured salmon sperm DNA; hybridization for 18-20 h at 40° C. in a solution containing 35% formamide, 5× SSC, 50 mM Tris-HCl (pH7.5), 5 mM EDTA, 0.02% PVP, 0.02% Ficoll, 0.2% BSA, 100 μg/ml salmon sperm DNA and 10% (wt/vol) dextran sulfate; followed by washing twice for 1 hour at 55° C. in a solution containing 2× SSC and 0.1% SDS.

Still other preferred derivative nucleotide sequences of the present invention are those capable of hybridizing to SEQ ID NO:1 under low stringency conditions including: incubation for 8 hours to overnight at 37° C. in a solution containing 20% formamide, 5× SSC, 50 mM sodium phosphate (pH 7.6), 5× Denhardt's solution, 10% dextran sulfate and 20 μg/ml denatured sheared salmon sperm DNA; hybridization in the same buffer for 18 to 20 hours; and washing of filters in 1× SSC at 37° C. for 1 hour.

As used herein, “percent nucleotide sequence identity”, means that percentage of nucleotides in a derivative nucleotide sequence identical to the nucleotides in the subject sequence (or specified portion thereof) after aligning the sequences and introducing gaps, if necessary, to achieve the maximum percent sequence identity as generated by the program WU-BLAST-2.0a19 (Altschul et al., J. Mol. Biol. (1997) 215:403-410; hereinafter referred to as “BLAST”) with all of the search parameters set to default values. The HSP S and HSP S2 parameters are dynamic values established by the program itself depending upon the composition of the particular sequence and composition of the particular database against which the sequence of interest is being searched. A percent nucleotide sequence identity value is determined by the number of matching identical nucleotides divided by the sequence length for which the percent identity is being reported.

The derivative dmCHT nucleic acids of the present invention possess at least 70% sequence identity, preferably at least 80% sequence identity, more preferably at least 85% sequence identity, still more preferably at least 90% sequence identity, and most preferably, at least 95% sequence identity with SEQ ID NO:1, or domain-encoding regions thereof.

In various embodiments of the present invention, derivative nucleic acids encode a polypeptide containing the dmCHT amino acid sequence of SEQ ID NO:2, or a fragment or derivative thereof of the present invention as described further herein below.

A derivative dmCHT nucleic acid, or fragment thereof, may have 100% sequence identity with SEQ ID NO:1 of the present invention, but still be a derivative thereof in the sense that it has one or more modifications at the base or sugar moiety, or phosphate backbone. Examples of such modifications are well known in the art (Bailey, Ullmann's Encyclopedia of Industrial Chemistry (1998), 6th ed. Wiley and Sons). Such derivatives may be utilized to provide modified stability or any other needed property.

Another type of derivative of the nucleic acid of the present invention may be a corresponding humanized sequence. By “humanized” sequence, the inventor herein means a nucleotide sequence in which one or more codons have been substituted by a codon or codons more commonly found in human genes. Preferably, a sufficient number of codons may be substituted such that a higher level of expression may be achieved in mammalian cells than would otherwise be achieved without those substitutions. The following list provides, for each amino acid, the calculated codon frequency (number in parentheses) in human genes for 1000 codons (Wada et al., Nucleic Acids Research (1990) 18(Suppl.): 2367-2411).

Human Codon Frequency per 1000 Codons

ARG: CGA (5.4), CGC (11.3), CGG (10.4), CGU (4.7), AGA (9.9), AGG (11.1)

LEU: CUA (6.2), CUC (19.9), CUG (42.5), CUU (10.7), UUA (5.3), UUG (11.0)

SER: UCA (9.3), UCC (17.7), UCG (4.2), UCU (13.2), AGC (18.7), AGU (9.4)

THR: ACA (14.4), ACC (23.0), ACG (6.7), ACU (12.7)

PRO: CCA(14.6),CCC(20.0),CCG(6.6),CCU(15.5)

ALA: GCA (14.0), GCC (29.1), GCG (7.2), GCU (19.6)

GLY: GGA (17.1), GGC (25.4), GGG (17.3), GGU (11.2)

VAL: GUA (5.9), GUC (16.3), GUG (30.9), GUU (10.4)

LYS: AAA (22.2), AAG (34.9)

ASN: AAC (22.6), AAU (16.6)

GLN: CAA (11.1), CAG (33.6)

HIS: CAC (14.2), CAU (9.3)

GLU: GAA (26.8), GAG (41.4)

ASP: GAC (29.0), GAU (21.7)

TYR: UAC (18.8), UAU (12.5)

CYS: UGC (14.5), UGU (9.9)

PHE: UUU (22.6), UUC (15.8)

ILE: AUA (5.8), AUC (24.3), AUU (14.9)

MET: AUG (22.3)

TRP: UGG (13.8)

TER: UAA (0.7), AUG (0.5), UGA (1.2)

As an example, a dmCHT nucleotide sequence wherein the glutamic acid (GLU) codon, GAA has been replaced with the codon GAG, more commonly found in human genes, may be considered a humanized dmCHT nucleotide sequence. A detailed discussion of humanization of nucleotide sequences is provided in U.S. Pat. No. 5,874,304 issued to Zolotukhin et al.

As will be apparent to those skilled in the art, other nucleic acid derivatives may be generated having codon(s) optimized for expression into specific organisms, such as yeast, bacteria and plants. This may permit one to engineer the expression of dmCHT polypeptides by employing specific codons, chosen according to the preferred codons used in highly expressed genes in that organism.

Nucleic acids encoding the amino acid sequence of SEQ ID NO:2, fragments or derivatives thereof of the present invention, may be obtained from an appropriate cDNA library prepared from any eukaryotic species that encodes CHT proteins such as vertebrates, preferably mammals (e.g. primate, porcine, bovine, feline, equine and canine species, etc.) and invertebrates, such as arthropods, particularly insects species (preferably Drosophila), acarids, crustacea, molluscs, nematodes and other worms.

An expression library may be constructed using known methods. For example, mRNA may be isolated to make cDNA that is ligated into a suitable expression vector for expression in a host cell into which it is introduced. Various screening assays may be employed to select for the gene or gene product (e.g. oligonucleotides of at least about 20 to 80 bases designed to identify the gene of interest, or labeled antibodies that specifically bind to the gene product). The gene and/or gene product may be recovered from the host cell by known techniques.

Polymerase chain reaction (PCR) may also be used to isolate nucleic acids of the dmCHT polypeptide of the present invention wherein oligonucleotide primers representing fragmentary sequences of interest amplify RNA or DNA sequences from a source such as a genomic or cDNA library (as described by Sambrook et al., supra). Further, degenerate primers for amplifying homologs from any species of interest may be used. Where a PCR product of appropriate size and sequence is obtained, it may be cloned and sequenced by standard techniques and employed as a probe for isolating a complete cDNA or genomic clone.

Fragmentary sequences of dmCHT nucleic acids may be synthesized by known methods. For example, oligonucleotides may be synthesized with an automated DNA synthesizer (such as those available from commercial suppliers including Biosearch, Novato, Calif.; Perkin-Elmer Applied Biosystems, Foster City, Calif.). Antisense RNA sequences may be produced intracellularly by transcription from an exogenous sequence, such as from vectors containing antisense dmCHT nucleotide sequences. Newly generated sequences may be identified and isolated by standard methods.

The isolated dmCHT nucleic acids, fragments and derivatives thereof of the present invention may be inserted into any appropriate cloning vector including, but not limited to, bacteriophages such as lambda derivatives, or plasmids such as PBR322, pUC plasmid derivatives and the Bluescript vector (Stratagene, San Diego, Calif.). Recombinant molecules may be introduced into host cells via transformation, transfection, infection, electroporation, etc., or into a transgenic animal such as a fly. The transformed cells may be cultured to generate large quantities of the dmCHT nucleic acids of the present invention. Suitable methods for isolating and producing the nucleotide sequences are well-known in the art (Sambrook et al., supra; DNA Cloning: A Practical Approach, Vol. 1, 2, 3, 4, (1995) Glover, ed., MRL Press, Ltd., Oxford, U.K.).

The nucleotide sequence encoding the dmCHT polypeptides, fragments or derivatives thereof of the present invention may be inserted into any appropriate expression vector for the transcription and translation of the inserted polypeptide-coding sequence. Alternatively, the necessary transcriptional and translational signals may be supplied by the native dmCHT gene and/or its flanking regions. A variety of host-vector systems may be employed to express the polypeptide-coding sequence including, but not limited to, mammalian cell systems infected with a virus (e.g. vaccinia virus, adenovirus, etc.), insect cell systems infected with a virus (e.g. baculovirus), microorganisms such as yeast containing yeast vectors, or bacteria transformed with bacteriophage, DNA, plasmid DNA, or cosmid DNA.

Expression of the dmCHT polypeptides, fragments and derivatives thereof of the present invention may be controlled by any suitable promoter/enhancer element. Further, a host cell strain may be selected which modulates the expression of the inserted sequences, or modifies and processes the gene product in the specific fashion desired. To detect expression of the dmCHT gene product, the expression vector may preferably contain a promoter operably linked to a dmCHT gene nucleic acid, one or more origins of replication and one or more selectable markers such as thymidine kinase activity or resistance to antibiotics. Alternatively, recombinant expression vectors may be identified by assaying for the expression of the dmCHT gene product based on the physical or functional properties of the dmCHT polypeptides, fragments and derivatives thereof of the present invention in in vitro assay systems such as immunoassays.

Where a recombinant expressing the dmCHT gene sequence is identified, the gene product may be isolated and purified by standard methods including, but not limited to: ion exchange, affinity and gel exclusion chromatography; centrifugation; differential solubility; and electrophoresis. The amino acid sequence may be deduced from the nucleotide sequence of a chimeric gene contained in the recombinant and may be synthesized by standard chemical methods (Hunkapiller et al., Nature (1984) 310:105-111). Alternatively, native dmCHT proteins may be purified from natural sources by standard methods such as immunoaffinity purification.

II. Drosophila melanogaster Choline Transporter (dmCHT) Polypeptides

The dmCHT polypeptides of the present invention contain an amino acid sequence given by SEQ ID NO:2, or fragments or derivatives thereof. The dmCHT polypeptide derivatives share a certain degree of sequence identity or sequence similarity with SEQ ID NO:2, or a fragment thereof.

As used herein, “percent amino acid sequence identity” means that percentage of amino acids in the derivative amino acid sequence identical to the amino acids in the subject sequence (or specified portion thereof) after aligning the sequences and introducing gaps, if necessary, to achieve the maximum percent sequence identity as generated by BLAST (Altschul et al., supra) using the same parameters discussed above for derivative nucleotide sequences. A percent amino acid sequence identity value may be determined by the number of matching identical amino acids divided by the sequence length for which the percent identity is being reported.

“Percent amino acid sequence similarity” may be determined by performing the same calculation as for determining percent amino acid sequence identity and including conservative amino acid substitutions in addition to identical amino acids in the computation. A conservative amino acid substitution is one in which an amino acid may be substituted for another amino acid having similar properties so that the folding or activity of the protein is not significantly affected.

The aromatic amino acids that may be substituted for each other are phenylalanine (PHE), tryptophan (TRP) and tyrosine (TYR). Interchangeable hydrophobic amino acids are leucine (LEU), isoleucine (ILE), methionine and valine (VAL). Interchangeable polar amino acids are glutamine (GLN) and asparagine (ASN). Interchangeable basic amino acids are arginine (ARG), lysine (LYS) and histidine (HIS). Interchangeable acidic amino acids are aspartic acid (ASP) and glutamic acid (GLU); and interchangeable small amino acids are alanine (ALA), serine (SER), cysteine (CYS), threonine (THR) and glycine (GLY).

In various embodiments of the present invention, a dmCHT polypeptide derivative may have at least 80% sequence identity or similarity, preferably at least 85%, more preferably at least 90%, and most preferably, at least 95% sequence identity or similarity with a contiguous stretch of at least 25 amino acids, preferably at least 50 amino acids, more preferably at least 100 amino acids and, in some cases, the entire length of SEQ ID NO:2. In another embodiment of the present invention, the dmCHT polypeptide derivative may contain a sequence that shares 100% similarity or identity with any contiguous stretch of at least 215 amino acids, preferably at least 220 amino acids, more preferably at least 225 amino acids and most preferably at least 230 amino acids of SEQ ID NO:2.

The fragments and derivatives of the dmCHT polypeptides of the present invention may preferably be “functionally active” meaning that the dmCHT polypeptide, fragments and derivatives thereof exhibit one or more functional activities associated with the full-length, wild-type dmCHT protein containing the amino acid sequence of SEQ ID NO:2. As one example, a fragment or derivative may possess antigenicity such that it can be used in immunoassays, for immunization, for inhibition of dmCHT activity, etc, as discussed further below regarding generation of antibodies to dmCHT proteins. Preferably, a functionally active dmCHT fragment or derivative displays one or more biological activities associated with dmCHT proteins, such as ion conductance.

Functionally active fragments include those fragments exhibiting one or more structural features of a dmCHT, such as multiple extracellular or intracellular domains. The functional activity of dmCHT polypeptides, derivatives and fragments thereof of the present invention may be assayed by various methods known to those skilled in the art (Current Protocols in Protein Science (1998) Coligan et al., eds., John Wiley & Sons, Inc., Somerset, N.J.). In one preferred method, described in detail herein below, a model organism such as Drosophila, may be used in genetic studies to assess the phenotypic effect of a fragment or derivative (i.e. a mutant dmCHT protein).

The dmCHT derivatives may be produced by various methods known to those skilled in the art. The manipulations that result in production of such derivatives may occur at the gene and/or protein level. For example, a cloned dmCHT gene sequence may be cleaved at appropriate sites with restriction endonuclease(s) (Wells et al., Philos. Trans. R. Soc. London SerA (1986) 317:415), followed by further enzymatic modification if desired, isolated, ligated in vitro and expressed to produce the desired derivative. Alternatively, a dmCHT gene may be mutated in vitro or in vivo to create and/or destroy translation, initiation and/or termination sequences, or to create variations in coding regions and/or to form new restriction endonuclease sites or to destroy preexisting ones and/or to facilitate further in vitro modification. A variety of techniques are known in the art such as chemical mutagenesis, in vitro site-directed mutagenesis (Carter et al., Nucl. Acids Res. (1986) 13:4331), use of TAB® linkers (available from Pharmacia and Upjohn, Kalamazoo, Mich.), etc.

At the protein level, manipulations include, but are not limited to, post translational modification, such as glycosylation, acetylation, phosphorylation, amidation, derivatization by known protecting/blocking groups, proteolytic cleavage and linkage to an antibody molecule or other cellular ligand. Any of numerous chemical modifications may be carried out by known techniques including, but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin, etc.). Derivative proteins may also be chemically synthesized by a peptide synthesizer, to introduce nonclassical amino acids or chemical amino acid analogs as substitutions or additions into the dmCHT protein sequence.

Chimeric or fusion proteins may be made containing the dmCHT polypeptides, fragments or derivatives thereof of the present invention preferably containing one or more structural or functional domains of the dmCHT protein joined at its amino- or carboxy-terminus via a peptide bond to an amino acid sequence of a different protein. Chimeric proteins may be produced by known methods such as recombinant expression of a nucleic acid encoding the protein containing the dmCHT-coding sequence joined in-frame to a coding sequence for a different protein, ligating the appropriate nucleotide sequences encoding the desired amino acid sequences to each other in the proper coding frame and expressing the chimeric product and protein synthetic techniques with a peptide synthesizer.

III. Drosophila melanogaster Choline Transporter (dmCHT) Gene Regulatory Elements

The dmCHT gene regulatory DNA elements such as enhancers or promoters may be employed to identify tissues, cells, genes and factors specifically controlling dmCHT protein production. Analyzing components specific to dmCHT protein function may lead to an understanding of how to manipulate such regulatory processes, especially for pesticide and therapeutic applications, as well as provide an understanding of how to diagnose dysfunction in those processes.

Gene fusions with the dmCHT regulatory elements may be made. For compact genes having relatively few and small intervening sequences, such as those described herein for Drosophila, the regulatory elements controlling spatial and temporal expression patterns may often be found in the DNA immediately upstream of the coding region, extending to the nearest neighboring gene. Regulatory regions may preferably be used to construct gene fusions where the regulatory DNAs are operably fused to a coding region for a reporter protein whose expression may be easily detected and such constructs may be introduced as transgenes into the animal of choice. An entire regulatory DNA region may be employed, or the regulatory region may be divided into smaller segments to identify sub-elements that might be specific for controlling expression a given cell type or stage of development. Reporter proteins preferred for the construction of such gene fusions include E. coli beta-galactosidase and green fluorescent protein (GFP). Those fusions may be detected readily in situ and therefore may be exploited in histological studies. Such gene fusions may also be employed in sorting cells expressing dmCHT proteins (O'Kane and Gehring PNAS (1987) 84(24):9123-9127; Chalfie et al., Science (1994) 263:802-805; and Cumberledge and Krasnow (1994) Methods in Cell Biology 44:143-159). Recombinase proteins, such as FLP or cre, may be used to control gene expression through site-specific recombination (Golic and Lindquist (1989) Cell 59(3):499-509; White et al., Science (1996) 271:805-807). Toxic proteins, such as the reaper and hid cell death proteins, may specifically ablate cells that normally express dmCHT proteins and may be employed in assessing the physiological function of the cells (Kingston, In Current Protocols in Molecular Biology (1998) Ausubel et al., John Wiley & Sons, Inc. sections 12.0.3-12.10) or in examining the function of those particular proteins specifically in cells that synthesize dmCHT proteins.

Alternatively, a binary reporter system may preferably be employed, as described herein below, wherein the dmCHT regulatory element may be operably fused to the coding region of an exogenous transcriptional activator protein, such as the GAL4 or tTA activators also described herein below, to create a dmCHT regulatory element “driver gene”. For the other half of the binary system, the exogenous activator controls a separate “target gene” containing a coding region of a reporter protein operably fused to a cognate regulatory element for the exogenous activator protein, such as UAS_{G} or a tTA-response element, respectively. One advantage of such a binary system is that a single driver gene construct may activate transcription from preconstructed target genes encoding different reporter proteins, each with its own uses as delineated above.

The dmCHT regulatory element-reporter gene fusions may also be exploited in tests of genetic interactions, wherein the goal is to identify those genes having a specific role in controlling the expression of dmCHT genes, or promoting the growth and differentiation of the tissues that express the dmCHT protein. dmCHT gene regulatory DNA elements also may be employed in protein-DNA binding assays to identify gene regulatory proteins controlling the expression of dmCHT genes. The gene regulatory proteins may be detected by a variety of methods probing specific protein-DNA interactions well known to those skilled in the art (Kingston, supra) including, but not limited to, in vivo footprinting assays based on protection of DNA sequences from chemical and enzymatic modification within living or permeabilized cells and in vitro footprinting assays based on protection of DNA sequences from chemical or enzymatic modification using protein extracts, nitrocellulose filter-binding assays and gel electrophoresis and mobility shift assays employing radioactively labeled regulatory DNA elements mixed with protein extracts.

dmCHT gene regulatory proteins may be purified by a combination of conventional and DNA-affinity purification techniques. Molecular cloning strategies may also identify proteins that specifically bind dmCHT gene regulatory DNA elements. For example, a Drosophila cDNA library in an expression vector may be screened for cDNAs encoding dmCHT gene regulatory element DNA-binding activity. In like manner, the yeast “one-hybrid” system may be used (Li and Herskowitz, Science (1993) 262:1870-1874; Luo et al., Biotechniques (1996) 20(4):564-568; Vidal et al., PNAS (1996) 93(19):10315-10320).

IV. Identification of Molecules Which Interact with dmCHT

A variety of methods may screen or identify molecules, such as proteins or other molecules, which interact with the dmCHT polypeptides, fragments and derivatives thereof of the present invention. Such assays may employ purified dmCHT polypeptides, cell lines or model organisms such as Drosophila and C. elegans, which have been genetically engineered to express the dmCHT polypeptides, fragments and derivatives thereof of the present invention. Suitable screening methodologies are well known in the art to test for proteins and other molecules that interact with the dmCHT gene and protein (see e.g., PCT International Publication No. WO 96/34099).

These newly identified interacting molecules may provide targets for pharmaceutical agents and/or pesticides. Any of a variety of exogenous molecules, both naturally occurring and/or synthetic (e.g., libraries of small molecules or peptides, or phage display libraries), may be screened for binding capacity as follows; the dmCHT polypeptide, fragment or derivative thereof of the present invention may be mixed with compounds under conditions conducive to binding, sufficient time allowed for any binding to occur and assays performed to test for bound complexes. Assays to identify interacting proteins may be performed by any method known in the art, for example, immunoprecipitation with an antibody that binds to the protein in a complex followed by analysis by size fractionation of the immunoprecipitated proteins (e.g. by denaturing or nondenaturing polyacrylamide gel electrophoresis), Western analysis, non-denaturing gel electrophoresis, etc.

A. Two-Hybrid Assay Systems

A preferred method for identifying proteins interacting with the dmCHT protein, fragment or derivative thereof of the present invention is a two-hybrid assay system or variation thereof (Fields and Song, Nature (1989) 340:245-246; U.S. Pat. No. 5,283,173; Brent and Finley, Annu. Rev. Genet. (1997) 31:663-704). The most commonly used two-hybrid assay system is performed using yeast. All such systems share three elements:

1) a gene that directs the synthesis of a “bait” protein fused to a DNA binding domain;

2) one or more “reporter” genes having an upstream binding site for the “bait”, and

3) a gene that directs the synthesis of a “prey” protein fused to an activation domain that activates transcription of the reporter gene.

For screening proteins that interact with the dmCHT polypeptides, fragments or derivatives thereof of the present invention, the “bait” may preferably be the dmCHT polypeptides, fragments or derivatives thereof of the present invention, preferably expressed as a fusion protein to a DNA binding domain. The “prey” protein is a protein to be tested for its ability to interact with the “bait” and may preferably be expressed as a fusion protein to a transcription activation domain. The “prey” proteins may be obtained from recombinant biological libraries expressing random peptides.

The “bait” fusion protein may preferably be constructed using any suitable DNA binding domain, such as the E. coli LexA repressor protein, or the yeast GAL4 protein (Bartel et al., BioTechniques (1993) 14:920-924, Chasman et al., Mol. Cell. Biol. (1989) 9:4746-4749; Ma et al., Cell (1987) 48:847-853; Ptashne et al., Nature (1990) 346:329-331).

The “prey” fusion protein may be constructed using any suitable activation domain such as GAL4, VP-16, etc. The “prey”s may preferably contain moieties such as nuclear localization signals (Ylikomi et al., EMBO J. (1992) 11:3681-3694; Dingwall and Laskey, Trends Biochem. Sci. Trends Biochem. Sci. (1991) 16:479-481) or epitope tags (Allen et al., Trends Biochem. Sci. Trends Biochem. Sci. (1995) 20:511-516) to facilitate isolation of the encoded proteins.

Preferably, a reporter gene having a detectable phenotype is chosen, thereby allowing cells expressing that gene to be selected by growth on appropriate medium (e.g. HIS3, LEU2 described by Chien et al., PNAS (1991) 88:9572-9582; and Gyuris et al., Cell (1993) 75:791-803). Other reporter genes, such as LacZ and GFP, allow cells expressing those genes to be visually screened (Chien et al., supra).

Although the preferred host for two-hybrid screening is yeast, the host cell in which the interaction assay and transcription of the reporter gene occurs may be any cell, including chicken, bacterial, or insect cells and mammalian cells, such as monkey, mouse, rat, human and bovine. Various vectors and host strains for expressing the two fusion protein populations in yeast may be employed (U.S. Pat. No. 5,468,614; Bartel et al., Cellular Interactions in Development (1993) Hartley, ed., Practical Approach Series xviii, IRL Press at Oxford University Press, New York, N.Y., pp. 153-179; and Fields and Sternglanz, Trends In Genetics (1994) 10:286-292). As an example of a mammalian system, interaction of activation tagged VP16 derivatives with a GAL4-derived “bait” drives expression of reporters that direct the synthesis of hygromycin B phosphotransferase, chloramphenicol acetyltransferase, or CD4 cell surface antigen (Fearon et al., PNAS (1992) 89:7958-7962). As another example, interaction of VP16-tagged derivatives with GAL4-derived “baits” drives the synthesis of SV40 T antigen, which in turn promotes the replication of the “prey” plasmid that carries an SV40 origin (Vasavada et al., PNAS (1991) 88:10686-10690).

The “bait” dmCHT gene and the “prey” library of chimeric genes may preferably be combined by mating the two yeast strains on solid or liquid media for a period of approximately 6-8 hours. The resulting diploids will contain both kinds of chimeric genes, i.e., the DNA-binding domain fusion and the activation domain fusion.

Transcription of the reporter gene may be detected by a linked replication assay in the case of SV40 T antigen (described by Vasavada et al., supra) or by immunoassay methods, preferably as described in Alam and Cook (Anal. Biochem. (1990)188:245-254). The activation of other reporter genes like URA3, HIS3, LYS2, or LEU2 enables cell growth in the absence of uracil, histidine, lysine, or leucine, respectively, and thereby serves as a selectable marker. Other types of reporters may be monitored by measuring a detectable signal; for example, GFP and lacZ have fluorescent and chromogenic gene products, respectively.

Where interacting proteins have been identified, the DNA sequences encoding the proteins may be isolated. In one method, the activation domain sequences or DNA-binding domain sequences, depending on the “prey” hybrid used, may be amplified, such as by PCR, using pairs of oligonucleotide primers specific for the coding region of the DNA binding domain or activation domain. Other known amplification methods may be employed, such as ligase chain reaction, use of Q replicase, or various other methods such as those described in Kricka et al., Molecular Probing, Blotting, and Sequencing (1995) Academic Press, New York, Chapter 1 and Table IX.

Where a shuttle (yeast to E. coli) vector is employed to express the fusion proteins, the DNA sequences encoding the proteins may be isolated by transformation of E. coli using the yeast DNA and recovering the plasmids from E. coli. Alternatively, the yeast vector may be isolated and the insert encoding the fusion protein subcloned into a bacterial expression vector for growth of the plasmid in E. coli.

A limitation of the two-hybrid system may occur where transmembrane portions of proteins in the “bait” or the “prey” fusions are used. Because most two-hybrid systems were designed to function by formation of a functional transcription activator complex within the nucleus, the use of transmembrane portions of the protein may interfere with proper association, folding and nuclear transport of “bait” or “prey” segments (Ausubel et al., supra; Allen et al., supra). As the dmCHT protein is a transmembrane protein, the inventor herein prefers that intracellular or extracellular domains be utilized herein for “bait” in a two-hybrid scheme.

B. Antibodies and Immunoassays

The dmCHT polypeptides, fragments and derivatives thereof of the present invention, such as those discussed above, may also function as immunogens in generating monoclonal or polyclonal antibodies and antibody fragments or derivatives (e.g. chimeric, single chain, Fab fragments).

For example, fragments of the dmCHT polpeptides of the present invention, preferably those identified as hydrophilic, may be used as immunogens for antibody production by art-known methods such as by hybridomas, production of monoclonal antibodies in germ-free animals (PCT International Publication No. WO99/02545), the use of human hybridomas (Cole et al., PNAS (1983) 80:2026-2030; Cole et al., in Monoclonal Antibodies and Cancer Therapy (1985) Alan R. Liss, pp. 77-96) and the production of humanized antibodies (Jones et al., Nature (1986) 321:522-525; U.S. Pat. No. 5,530,101).

In a one embodiment of the present invention, the dmCHT polypeptide fragments provide specific antigens and/or immunogens, especially if coupled to carrier proteins. For example, peptides of the present invention may be covalently coupled to keyhole limpet antigen (KLH) and the conjugate emulsified in Freund's complete adjuvant. Laboratory rabbits may be immunized according to conventional protocol and bled. The presence of specific antibodies may be assayed by solid phase immunosorbent assays employing immobilized corresponding polypeptides. Specific activity or function of the antibodies produced may be determined by convenient in vitro, cell-based, or in vivo assays: e.g. in vitro binding assays, etc. Binding affinity may be assayed by determining the equilibrium constants of antigen-antibody association, preferably at least about 10⁷ M⁻¹, more preferably at least about 10⁸ M⁻¹ and most preferably at least about 10⁹ M⁻¹.

Immunoassays may identify proteins interacting with or binding to the dmCHT polypeptides, fragments or derivatives thereof of the present invention. Various assays are available for testing the ability of a protein to bind to or compete with binding to a wild-type dmCHT protein or for binding to an anti-dmCHT protein antibody including, but not limited to, radioimmunoassays, ELISA (enzyme linked immunosorbent assay), immunoradiometric assays, gel diffusion precipitin reactions, immunodiffusion assays, in situ immunoassays (e.g., using colloidal gold, enzyme or radioisotope labels), Western blots, precipitation reactions, agglutination assays (e.g., gel agglutination assays, hemagglutination assays), complement fixation assays, immunofluorescence assays, protein A assays and immunoelectrophoresis assays.

C. Identification of Potential Pesticide or Drug Targets

The dmCHT genes or dmCHT interacting genes identified by methods of the present invention may be assessed as potential pesticide or drug targets, or as potential biopesticides. Further, transgenic plants expressing the dmCHT polypeptides, fragments or derivatives thereof of the present invention may be tested for activity against insect pests (Estruch et al., Nat. Biotechnol (1997) 15(2):137-141).

As used herein, the term pesticide includes, but is not limited to, chemicals, biological agents and other compounds that kill, paralyze, sterilize or otherwise disable pest species in the areas of agricultural crop protection, human and animal health.

Exemplary pest species include parasites and disease vectors such as mosquitoes, fleas, ticks, parasitic nematodes, chiggers, mites, etc. Pest species also include those eradicated for aesthetic and hygienic purposes (e.g. ants, cockroaches, clothes moths, flour beetles, etc.), home and garden applications and protection of structures (including, but not limited to, wood boring pests such as termites and marine surface fouling organisms).

Pesticides may also include the traditional small organic molecule pesticides (typified by compound classes such as the organophosphates, pyrethroids, carbamates and organochlorines, benzoylureas, etc.), proteinaceous toxins such as the Bacillus thuringiensis Crytoxins (Gill et al., Annu Rev Entomol (1992) 37:615-636) and Photorabdus luminescens toxins (Bowden et al., Science (1998) 280:2129-2132) and nucleic acids such as dmCHT dsRNA or antisense nucleic acids that interfere with dmCHT activity.

Pesticides may be delivered by a any of a variety of means including, but not limited to, direct application to pests and/or to the pests' food source and toxic proteins and pesticidal nucleic acids (e.g. dsRNA) may be administered using biopesticidal methods, for example, by viral infection with nucleic acid or by transgenic plants modified to produce interfering nucleotide sequences or to encode the toxic protein, which are ingested by plant-eating pests.

Putative pesticides, drugs and other molecules of interest may be applied onto whole insects, nematodes and other small invertebrate metazoans and the ability of such compounds to modulate (e.g. block or enhance) dmCHT activity observed. Alternatively, the effect of various compounds on dmCHTs may be assayed in cells engineered to express one or more dmCHTs and associated proteins.

1. Assays of Compounds on Insects

Potential insecticidal compounds may be administered to insects in a variety of ways, including, but not limited to, orally (such as by addition to synthetic diet or by application to plants or prey to be consumed by the test organism), topically (such as by spraying, direct application of compound to animal or allowing animal to contact a treated surface) and by injection. Insecticides are often very hydrophobic molecules which therefore must be dissolved in organic solvents that may be allowed to evaporate in the case of methanol or acetone, or at low concentrations may be included to facilitate uptake (e.g. ethanol, dimethyl sulfoxide).

The first step in an insect assay may preferably be the determination of the minimal lethal dose (“MLD”) on the insects following a chronic exposure to the compound(s). The compound(s) may preferably be diluted in DMSO and applied to a food surface bearing 0-48 hour old embryos and larvae. In addition to determining the MLD, this allows the observation of the fraction of eggs that hatch, of the behavior of the larvae, such as movement and feeding compared to untreated larvae, of the fraction that survive to pupate and of the fraction that eclose (emergence of the adult insect from puparium). In addition, larvae may be dissected to look for obvious morphological defects. Based on these results, more detailed assays with shorter exposure times may preferably be designed.

In an acute assay, the compound(s) may preferably be applied to the food surface for embryos, larvae, or adults and the animals observed after 2 hours and following an overnight incubation. For application on embryos, defects in development and the percent surviving to adulthood may be determined. For larvae, defects in behavior, locomotion and molting may be observed. For application on adults, behavior and neurological defects may be observed and effects on fertility noted.

For a chronic exposure assay, adults may be placed on vials containing the compound(s) for 48 hours, transferred to a clean container and observed for fertility, neurological defects death.

2. Assays of Compounds on Worms

In a worm assay, the compound(s) to be tested may be dissolved in DMSO or other organic solvent, mixed with a bacterial suspension at various test concentrations, preferably the OP50 strain of bacteria (Brenner, Genetics (1974) 110:421-440) and supplied as food to the worms. The population of worms to be treated may be synchronized larvae (Sulston and Hodgkin, in The nematode C. elegans (1988), supra), adults or a mixed-stage population of animals.

Adult and larval worms may be treated with different concentrations of the compound(s), ranging preferably from about 1 mg/ml to about 0.001 mg/ml. Behavioral aberrations, such as decreased motility and growth and morphological aberrations, sterility and death may be examined in both acutely and chronically treated adult and larval worms. For the acute assay, larval and adult worms may be examined immediately after application of the compound(s) and re-examined periodically (every 30 minutes) for 5-6 hours. Chronic or long-term assays may be performed on worms and the behavior of the treated worms may be examined every 8-12 hours for 4-5 days. In some circumstances, it may be necessary to reapply the compound(s) to the treated worms every 24 hours for maximal effect.

3. Assay of Compounds Using Cell Culture

Compounds modulating (e.g. blocking or enhancing) dmCHT activity may also be assayed in cultured cells. For example, various compounds added to cells expressing the dmCHT polypeptides, fragments and derivatives thereof of the present invention may be screened for the ability to modulate the activity of dmCHT genes based upon measurements of choline transport. Assays for changes in choline uptake may be performed on cultured cells expressing endogenous normal or mutant dmCHTs. Such studies also may be performed on cells transfected with vectors capable of expressing the dmCHTs or functional domains of one of the dmCHTs in normal or mutant form. In addition, cells may be cotransfected with genes encoding dmCHT proteins to enhance the signal measured in such assays.

For example, Xenopus oocytes may be injected with normal or mutant dmCHT. Changes in dmCHT-related or dmCHT-mediated transport activity may be measured by two-microelectrode voltage-clamp recordings in oocytes and/or by rate of uptake of radioactive choline molecules (Arriza et al., J. Neurosci.(1994) 14:5559-5569; Arriza et al., J. Biol. Chem. (1993) 268:15329-15332; Mbungu et al., Archives of Biochemistry and Biophysics (1995) 318:489-497). A battery of compounds, particularly potential pesticides or drugs, may be screened by such procedures. The selectivity of a material for dmCHT may be determined by testing the effect of the compound using cells expressing dmCHT and comparing the results with that obtained using cells not expressing dmCHT (see U.S. Pat. Nos. 5,670,335 and 5,882,873).

Compounds selectively modulating the dmCHT of the present invention may be identified as potential pesticide and drug candidates having dmCHT specificity. Identification of small molecules and compounds as potential pesticides or pharmaceutical compounds from large chemical libraries may be facilitated by high-throughput screening (HTS) methods (Bolger, Drug Discovery Today (1999) 4:251-253). A number of the assays described herein may lend themselves to high-throughput screening. For example, cells or cell lines expressing wild type or mutant dmCHT protein or its fragments and a reporter gene, may be subjected to compounds of interest and depending on the reporter genes, interactions may be measured using a variety of methods such as color detection, fluorescence detection (e.g. GFP), autoradiography, scintillation analysis, etc.

V. dmCHT Nucleic Acids as Biopesticides

The dmCHT nucleic acids, fragments and derivatives thereof of the present invention, such as antisense sequences or double-stranded RNA (dsRNA), may inhibit dmCHT function and thereby may act as biopesticides. Methods of using dsRNA interference are described in published PCT International Publication No. WO 99/32619. Such biopesticides may contain the dmCHT nucleic acids, fragments and derivatives thereof of the present invention, an expression construct capable of expressing the dmCHT nucleic acids, fragments and derivatives thereof of the present invention or organisms transfected with the expression construct. The biopesticide(s) may be applied directly to plant parts or to the soil surrounding the plant (e.g. to access plant parts growing beneath ground level) or directly onto the pest. The biopesticides containing the dmCHT nucleic acids, fragments and derivatives of the present invention may preferably be applied in an “effective amount” i.e., an amount sufficient to be lethal to the pest.

Biopesticides containing the dmCHT nucleic acids, fragments and derivatives thereof of the present invention may be prepared in any suitable vector for delivery to a plant or animal. Preferred vectors for generating plants expressing the dmCHT nucleic acids of the present invention include, but are not limited to, Agrobacterium tumefaciens Ti plasmid-based vectors (Horsch et al., Science (1984) 233:496-89; Fraley et al., Proc. Natl. Acad. Sci. USA (1983) 80:4803) and recombinant cauliflower mosaic virus (Hohn et al., 1982, In Molecular Biology of Plant Tumors, Academic Press, New York, pp 549-560; U.S. Pat. No. 4,407,956 to Howell). Retrovirus-based vectors may also be useful for the introduction of genes into vertebrate animals (Burns et al., Proc. Natl. Acad. Sci. USA (1993) 90:8033-37).

Biopesticides incorporating the dmCHT nucleic acids, fragments and derivatives thereof of the present invention may also include an appropriate carrier. The carrier may preferably assist in delivery of the biopesticide to the target site. The choice of carrier, therefore, depends upon the habitat of the pest, the type of food consumed by the pest, and the physical characteristics of the biopesticide. The biopesticide preferably should be substantially soluble or dispersible in the carrier and the carrier should preferably be physiologically acceptable and/or compatible with invertebrates, plants, fish and other vertebrate organisms.

Preferred carriers may help the biopesticide adhere to plant leaves and other food sources consumed by the target pest. The carrier may also assist the biopesticide in adhering and/or penetrating the external skeletons, shells or cuticle of invertebrates. The carrier may also constitute a food source for the pest and may be formulated into a liquid or powdered mixture for spray delivery, or as granules or liposomes as needed. The carrier may further include a surface-active agent(s). Preferred surface-active agents will readily disperse in the carrier and increase the adherence and/or penetration of the carrier and biopesticide to the target site.

The biopesticide may also further include a chemical pesticide(s) as required by the particular pest to be controlled or eradicated. Many of the chemical pesticides currently in use provide less than total control or eradication of the pest(s), therefore, resistant or surviving larvae or adults simultaneously or subsequently exposed biopesticide may yield an increased mortality.

Transgenic insects may be generated by a transgene containing a dmCHT gene operably fused to an appropriate inducible promoter. For example, a tTA-responsive promoter may direct expression of the dmCHT polypeptide, fragment or derivative thereof of the present invention at an appropriate time in the life cycle of the insect. In this manner, the efficacy of a compound as an insecticide may be tested in, for example, the larval phase of the life cycle (i.e. when feeding does the greatest damage to crops). Vectors for the introduction of genes into insects include, but are not limited to, P element (Rubin and Spradling, Science (1982) 218:348-53; U.S. Pat. No. 4,670,388), “hermes” (O'Brochta et al., Genetics (1996) 142:907-914), “minos” (U.S. Pat. No. 5,348,874), “mariner” (Robertson, Insect Physiol. (1995) 41:99-105) and “sleeping beauty” (lvics et al., Cell (1997) 91(4):501-510), “piggyBac” (Thibault et al., Insect Mol Biol (1999) 8(1):119-23) and “hobo” (Atkinson et al., Proc. Natl. Acad. Sci. U.S.A. (1993) 90:9693-9697).

Recombinant virus systems for expression of toxic proteins in infected insect cells are well known and include Semliki Forest virus (DiCiommo and Bremner, J. Biol. Chem. (1998) 273:18060-66), recombinant sindbis virus (Higgs et al., Insect Mol. Biol. (1995) 4:97-103; Seabaugh et al., Virology (1998) 243:99-112), recombinant pantropic retrovirus (Matsubara et al., Proc. Natl. Acad. Sci. USA (1996) 93:6181-85; Jordan et al., Insect Mol. Biol. (1998) 7:215-22) and recombinant baculovirus (Cory and Bishop, Mol. Biotechnol. (1997) 7(3):303-13; U.S. Pat. No. 5,470,735; U.S. Pat. No. 5,352,451; U.S. Pat. No. 5, 770, 192; U.S. Pat. No. 5,759,809; U.S. Pat. No. 5,665,349; and U.S. Pat. No. 5,554,592).

VI. Generation and Genetic Analysis of Animals and Cell Lines with Altered Expression of dmCHT Gene

Both genetically modified animal models (i.e. in vivo models) such as C. elegans and Drosophila and in vitro models such as genetically modified cell lines expressing or mis-expressing dmCHT pathway genes may preferably be employed in the functional analysis of dmCHT proteins. Model systems exhibiting detectable phenotypes, may preferably be utilized in the identification and/or characterization of dmCHT pathway genes or other genes of interest and/or phenotypes associated with the mutation or mis-expression of dmCHT pathway protein.

The term “mis-expression” as used herein encompasses mis-expression due to gene mutations. Thus, a mis-expressed dmCHT pathway protein may be one having an amino acid sequence that differs from wild-type, i.e. it is a derivative of the normal protein. A mis-expressed dmCHT pathway protein may also be one in which one or more amino acids have been deleted and thus is a “fragment” of the normal protein. As used herein, “mis-expression” also is meant to include ectopic expression (e.g. by altering the normal spatial or temporal expression), over-expression (e.g. by multiple gene copies), underexpression, non-expression (e.g. by gene knockout or blocking expression that would otherwise normally occur) and further, expression in ectopic tissues. As used in the following discussion concerning in vivo and in vitro models and elsewhere in the specification, the term “gene of interest” refers to a dmCHT pathway gene, or any other gene involved in regulation or modulation, or downstream effector of the dmCHT pathway.

The in vivo and in vitro models may be genetically engineered or modified so that the models:

1) have deletions and/or insertions of one or more dmCHT pathway genes;

2) harbor interfering RNA sequences derived from dmCHT pathway genes;

3) have had one or more endogenous dmCHT pathway genes mutated (e.g. contain deletions, insertions, rearrangements, or point mutations in dmCHT gene or other genes in the pathway); and/or

4) contain transgenes for mis-expression of wild-type or mutant forms of such genes.

Such genetically modified in vivo and in vitro models may be employed in the identification of genes and proteins involved in the synthesis, activation, control, etc. of the dmCHT pathway gene and/or gene products and downstream effectors of dmCHT function, genes regulated by dmCHT, etc. The newly identified genes may also constitute possible pesticide targets as judged by animal model phenotypes such as non-viability, block of normal development, defective feeding, defective movement, or defective reproduction.

Such model systems may also be employed for testing potential pesticidal or pharmaceutical compounds that interact with the dmCHT pathway, for example by administering the compound(s) to the model system by any suitable method (e.g. direct contact, ingestion, injection, etc.) and observing any changes in phenotype, for example defective movement, lethality, etc. Various genetic engineering and expression modification methods may be applied which are well-known in the art, including chemical mutagenesis, transposon mutagenesis, antisense RNAi, dsRNAi and transgene-mediated mis-expression.

A. Generating Loss-of-Function Mutations by Mutagenesis

Loss-of-function mutations in an invertebrate metazoan dmCHT gene may be generated by any of several mutagenesis methods known in the art (Ashburner, In Drosophila melanogaster: A Laboratory Manual (1989) , Cold Spring Harbor, N.Y., Cold Spring Harbor Laboratory Press: pp. 299-418; Fly pushing: The Theory and Practice of Drosophila melanogaster Genetics (1997) Cold Spring Harbor Press, Plainview, N.Y.; The nematode C. elegans (1988) Wood, Ed., Cold Spring Harbor Laboratory Press, Cold Spring harbor, N.Y.). Techniques for producing mutations in a gene or genome include, but are not limited to, use of radiation (e.g., X-ray, UV, or gamma ray), chemicals (e.g., EMS, MMS, ENU, formaldehyde, etc.) and insertional mutagenesis by mobile elements including dysgenesis induced by transposon insertions, or transposon-mediated deletions, for example, male recombination, as described below, use of transposons (e.g., P element, EP-type “overexpression trap” element, mariner element, piggyBac transposon, hermes, minos, sleeping beauty, etc.) to misexpress genes, gene targeting by homologous recombination, antisense, double-stranded RNA interference, peptide and RNA aptamers, directed deletions, homologous recombination, dominant negative alleles and intrabodies.

Transposon insertions lying adjacent to a gene of interest may be exploited in generating deletions of flanking genomic DNA, which if induced in the germline, are stably propagated in subsequent generations. The applicability of this technique in generating deletions has been demonstrated and is well-known in the art. One version of this technique employing collections of P element transposon induced recessive lethal mutations (P lethals) is particularly preferred for the rapid identification of novel, essential genes in Drosophila (Cooley et al., Science (1988) 239:1121-1128; Spralding et al., PNAS (1995) 92:0824-10830). Because the sequence of the P elements are known, the genomic sequence flanking each transposon insert may be determined either by plasmid rescue (Hamilton et al., PNAS (1991) 88:2731-2735) or by inverse polymerase chain reaction (Rehm, http://www.fruitfly.org/methods/). A more recent version of the transposon insertion technique in male Drosophila using P elements is known as P-mediated male recombination (Preston and Engels, Genetics (1996) 144:1611-1638).

Gene targeting approaches with homologous recombination have proven to be successful in Drosophila (Rong and Golic, Science (2000) 288:2013-20018) and may provide a general method of generating directed mutations in any gene-of-interest. These methods employ broken-ended extrachromosomal DNA, created in vivo, to produce homology-directed changes in a target locus. First, a “targeting construct” may be designed for the gene-of-interest which allows the replacement of the normal endogenous gene with a specifically designed mutation, such as a deletion, insertion or point mutation, via homologous recombination. The targeting construct may be carried in an appropriate transposon-mediated transgenesis vector (e.g. P element-, piggyBac-, hermes-, minos-, or mariner-based vectors) which inserts the targeting construct randomly within the genome of the organism.

The targeting construct may be converted to a recombinogenic extrachromosomal form by inducing the expression of separate transgenes encoding a site-specific recombinase (e.g. FLP, cre, Kw, etc.) which excises the targeting construct and a rare-cutting site-specific endonuclease (e.g. Scel, Crel, HO, etc.) that generates recombinogenic ends which direct homologous recombination and gene replacement of the endogenous locus. Although this method has thus far only been shown to work in Drosophila, the inventor herein believes that it may have application to worms, other animals, plants, algae etc.

1. Generating Loss-of-Function Phenotypes by RNA-Based Methods

The dmCHT genes may be identified and/or characterized by generating loss-of-function phenotypes in animals of interest through RNA-based methods, such as antisense RNA (Schubiger and Edgar, Methods in Cell Biology (1 994) 44:697-713). One form of the antisense RNA method involves the injection of embryos with an antisense RNA that is partially homologous to the gene of interest, in case of the present invention, the dmCHT gene.

Another form of the antisense RNA method involves expression of an antisense RNA partially homologous to the gene of interest by operably joining a portion of the gene of interest in the antisense orientation to a powerful promoter that may drive the expression of large quantities of antisense RNA, either generally throughout the animal or in specific tissues. Antisense RNA-generated loss-of-function phenotypes have been reported previously for several Drosophila genes including cactus, pecanex and Krüppel (LaBonne et al., Dev. Biol. (1989) 136(1):1-16; Schuh and Jackle, Genome (1 989) 31(1):422-425; Geisler et al., Cell (1992) 71(4):613-621).

Loss-of-function phenotypes may also be generated by cosuppression methods (Bingham Cell (1997) 90(3):385-387; Smyth, Curr. Biol. (1997) 7(12):793-795; Que and Jorgensen, Dev. Genet. (1998) 22(1):100-109). Cosuppression is a phenomenon of reduced gene expression produced by expression or injection of a sense strand RNA corresponding to a partial segment of the gene of interest. Cosuppression effects have been employed extensively in plants and C. elegans to generate loss-of-function phenotypes and there is at least one report of cosuppression in Drosophila, wherein reduced expression of the Adh gene was induced from a white-Adh transgene by cosuppression methods (Pal-Bhadra et al., Cell (1997) 90(3):479-490).

Another method for generating loss-of-function phenotypes is by double-stranded RNA interference (dsRNAi). This method is based on the interfering properties of double-stranded RNA derived from the coding regions of gene and has proven to be of great utility in genetic studies of C. elegans (Fire et al., Nature (1998) 391:806-811) and has also be used to generate loss-of-function phenotypes in Drosophila (Kennerdell and Carthew, Cell (1998) 95:1017-1026; Misquitta and Patterson PNAS (1999) 96:1451-1456). In one example of this method, complementary sense and antisense RNAs derived from a substantial portion of a gene of interest, such as dmCHT gene, may be synthesized in vitro. The resulting sense and antisense RNAs may be annealed in an injection buffer and the double-stranded RNA injected or otherwise introduced into animals (such as in the animal's food or by soaking in buffer containing the RNA). Progeny of the injected animals may be inspected for phenotypes of interest (PCT International Publication No. WO99/32619).

In another embodiment of this method, the dsRNA may be delivered by bathing the animal in a solution containing a sufficient concentration of the dsRNA. In still another embodiment of the method, dsRNA derived from dmCHT genes may be generated in vivo by simultaneous expression of both sense and antisense RNA from appropriately positioned promoters operably fused to dmCHT sequences in both sense and antisense orientations. In yet another embodiment of the method, the dsRNA may be delivered to the animal by engineering expression of dsRNA within cells of a second organism that serves as food for the animal, for example engineering expression of dsRNA in E. coli bacteria which are fed to C. elegans, or engineering expression of dsRNA in baker's yeast which are fed to Drosophila, or engineering expression of dsRNA in transgenic plants which are fed to plant eating insects such as Leptinotarsa or Heliothis.

Recently, RNAi has successfully inhibited expression of targeted proteins in cultured Drosophila cells (Dixon lab, University of Michigan, Clemens et al. PNAS, Jun. 6, 2000, vol. 97, no. 12, pp. 6499-6503). Thus, cell lines in culture may be manipulated by RNAi to perturb and study the function of dmCHT pathway components and to validate the efficacy of therapeutic or pesticidal strategies that involve the manipulation of this pathway.

2. Generating Loss-of-Function Phenotypes by Peptide and RNA Aptamers

Another method for generating loss-of-function phenotypes is by peptide aptamers, which are peptides or small polypeptides that act as dominant inhibitors of protein function. Peptide aptamers specifically bind to target proteins, blocking their function ability (Kolonin and Finley, PNAS (1998) 95:14266-14271). Thus, due to the highly selective nature of peptide aptamers, not only may a specific protein be targeted, but also specific functions of that protein (e.g. ion transport function) may be studied. Further, peptide aptamers may be expressed in a controlled fashion by promoters which regulate expression in a temporal, spatial or inducible manner. Because peptide aptamers act dominantly, proteins for which loss-of-function mutants are not available may be analyzed therewith.

Peptide aptamers that bind with high affinity and specificity to a target protein may be isolated by a variety of techniques known in the art. In one method, peptide aptamers may be isolated from random peptide libraries by yeast two-hybrid screens (Xu et al., PNAS (1997) 94:12473-12478). Peptide aptamers may also be isolated from phage libraries (Hoogenboom et al., Immunotechnology (1998) 4:1-20) or chemically generated peptides/libraries.

RNA aptamers are specific RNA ligands for proteins, which may specifically inhibit protein function of the gene (Good et al., Gene Therapy (1997) 4:45-54; Ellington. et al., Biotechnol. Annu. Rev. (1995) 1:185-214). In vitro selection methods may be used to identify RNA aptamers having a selected specificity (Bell et al., J. Biol. Chem. (1998) 273:14309-14314). It has been demonstrated that RNA aptamers may inhibit protein function in Drosophila (Shi et al., Proc. Natl. Acad. Sci USA (19999) 96:10033-10038). Therefore, RNA aptamers may be employed to decrease expression of the dmCHT proteins, fragments or derivatives thereof of the present invention, or of a protein that interacts therewith.

Transgenic animals may be generated to test peptide or RNA aptamers in vivo (Kolonin, M G, and Finley, R L, Genetics, 1998 95:4266-4271). For example, transgenic Drosophila lines expressing the desired aptamers may be generated by P element mediated transformation (discussed below). The phenotypes of the progeny expressing the aptamers may be characterized.

3. Generating Loss of Function Phenotypes with Intrabodies

Intracellularly expressed antibodies, or intrabodies, are single-chain antibody molecules designed to specifically bind and inactivate target molecules inside cells. Intrabodies have been used in cell assays and in whole organisms such as Drosophila (Chen et al., Hum. Gen. Ther. (1994) 5:595-601; Hassanzadeh et al., Febs Lett. (1998) 16(1, 2):75-80 and 81-86). Inducible expression vectors may be constructed with intrabodies that react specifically with the dmCHT polypeptides, fragments and derivatives thereof of the present invention. Those vectors may be introduced into model organisms and studied in the same manner as described above for aptamers.

4. Transgenesis

Transgenic animals may be created containing gene fusions of the coding regions of the dmCHT gene (from either genomic DNA or cDNA) or genes modified to encode antisense RNAs, cosuppression RNAs, interfering dsRNA, RNA aptamers, peptide aptamers, or intrabodies operably joined to a specific promoter and transcriptional enhancer whose regulation has been well characterized, preferably heterologous promoters/enhancers (i.e. promoters/enhancers that are non-native to the dmCHT pathway genes being expressed).

Methods are well known in the art for incorporating exogenous nucleotide sequences into the genome of animals or cultured cells to create transgenic animals or recombinant cell lines. For invertebrate animal models, most such methods involve the use of transposable elements. There are several suitable transposable elements which incorporate nucleotide sequences into the genome of model organisms. Transposable elements may be particularly applicable for inserting sequences into a gene of interest so that the encoded protein is not properly expressed, thereby creating a “knock-out” animal having a loss-of-function phenotype. Techniques are well-established for the use of P element in Drosophila (Rubin and Spradling, Science (1982) 218:348-53; U.S. Pat. No. 4,670,388) and Tc1 in C. elegans (Zwaal et al., Proc. Natl. Acad. Sci. U.S.A. (1993) 90:7431-7435; and Caenorhabditis elegans: Modern Biological Analysis of an Organism (1995) Epstein and Shakes, Eds.). Other Tc1-like transposable elements may be used such as minos, mariner and sleeping beauty. Additionally, transposable elements that function in a variety of species, have been identified, such as PiggyBac (Thibault et al., Insect Mol Biol (1999) 8(1):119-23), hobo and hermes.

P elements, or marked P elements, are preferred by the inventor herein for the isolation of loss-of-function mutations in Drosophila dmCHT genes. This is because of the precise molecular mapping of those genes, depending on the availability and proximity of preexisting P element insertions for use as a localized transposon source (Hamilton and Zinn, Methods in Cell Biology (1994) 44:81-94; and Wolfner and Goldberg, Methods in Cell Biology (1994) 44:33-80).

Preferably, modified P elements containing one or more elements allowing detection of animals possessing the P element may be employed. Marker genes may preferably be utilized that affect the eye color of Drosophila, such as derivatives of the Drosophila white or rosy genes (Rubin and Spradling, Science (1982) 218(4570):348-353; and Klemenz et al., Nucleic Acids Res. (1987) 15(10):3947-3959). However, in principle, any gene causing a reliable and easily scored phenotypic change in transgenic animals may function as a marker. Various other markers include, but are not limited to, bacterial plasmid sequences having selectable markers such as ampicillin resistance (Steller and Pirrotta, EMBO. J. (1985) 4:167-171); and lacZ sequences fused to a weak general promoter to detect the presence of enhancers with a developmental expression pattern of interest (Bellen et al., Genes Dev. (1989) 3(9):1288-1300). Other examples of marked P elements useful for mutagenesis have been reported (Nucleic Acids Research (1998) 26(1):85-88; and http://flybase.bio.indiana.edu).

A preferred method of transposon mutagenesis in Drosophila employs the “local hopping” method described by Tower et al. (Genetics (1993) 133:347-359). Each new P insertion line may be tested molecularly for transposition of the P element into the gene of interest (e.g. dmCHT) by assays based on PCR. For each reaction, one PCR primer may be employed that is homologous to sequences contained within the P element and a second primer that is homologous to the coding region or flanking regions of the gene of interest. Products of the PCR reactions may be detected by agarose gel electrophoresis. The sizes of the resulting DNA fragments reveal the site of P element insertion relative to the gene of interest. Alternatively, Southern blotting and restriction mapping with DNA probes derived from genomic DNA or cDNAs of the gene of interest may be utilized to detect transposition events that rearrange the genomic DNA of the gene. P transposition events that map to the gene of interest may be assessed for phenotypic effects in heterozygous or homozygous mutant Drosophila.

In another embodiment, Drosophila lines carrying P insertions in the gene of interest may be used to generate localized deletions using known methods (Kaiser, Bioassays (1990) 12(6):297-301; Harnessing the power of Drosophila genetics, In Drosophila melanogaster: Practical Uses in Cell and Molecular Biology, Goldstein and Fyrberg, Eds., Academic Press, Inc. San Diego, Calif.). This method is particularly preferred if no P element transpositions are found which disrupt the gene of interest. Briefly, flies containing P elements inserted near the gene of interest may be exposed to a further round of transposase to induce excision of the element. Progeny in which the transposon has excised may be identified by loss of the eye color marker associated with the transposable element. The resulting progeny will include flies with either precise or imprecise excision of the P element, wherein the imprecise excision events often result in deletion of genomic DNA neighboring the site of P insertion. Such progeny may be screened by molecular techniques to identify deletion events that remove genomic sequence from the gene of interest and assessed for phenotypic effects in heterozygous and homozygous mutant Drosophila.

A transgenesis system has been described by Berghammer et al., (Nature (1999) 402:370-371) that may have universal applicability in all eye-bearing animals and which has been proven effective in delivering transgenes to diverse insect species. This system includes an artificial promoter active in eye tissue of all animal species, preferably containing three Pax6 binding sites positioned upstream of a TATA box (3×P3; Sheng et al., Genes Devel. (1997) 11:1122-1131), a strong and visually detectable marker gene, such as GFP or other autofluorescent protein genes (Pasher et al., Gene (1992) 111:229-233; U.S. Pat. No. 5,491,084) and promiscuous vectors capable of delivering transgenes to a broad range of animal species. Examples of promiscuous vectors include transposon-based vectors derived from hermes, PiggyBac, or mariner and vectors based on pantropic VSV_{G}-pseudotyped retroviruses (Burns et al., In Vitro Cell Dev Biol Anim (1996) 32:78-84; Jordan et al., Insect Mol Biol (1998) 7: 215-222; U.S. Pat. No. 5,670,345). Because the same transgenesis system may be used in a variety of phylogenetically diverse animals, therefore, comparative functional studies may be greatly facilitated which may be especially helpful in evaluating new applications to pest management.

In C. elegans, Tc1 transposable element may be employed for directed mutagenesis of a gene of interest. A Tc1 library may preferably be prepared by the methods of Zwaal et al., supra and Plasterk, supra, using a strain wherein the Tc1 transposable element is highly mobile and present in a high copy number. The library may preferably be screened for Tc1 insertions in the region of interest using PCR with one set of primers specific for Tc1 sequence and one set of gene-specific primers. The C. elegans strains containing Tc1 transposon insertions within the gene of interest may be isolated.

In addition to creating loss-of-function phenotypes, transposable elements may be employed to incorporate the gene of interest, mutant or derivative thereof, as an additional gene into any region of an animal's genome resulting in mis-expression (including over-expression) of the gene. A preferred vector designed specifically for mis-expression of genes in transgenic Drosophila derived from pGMR (Hay et al., Development (1994) 120:2121-2129) is 9 Kb long and includes: an origin of replication for E. coli; an ampicillin resistance gene; P element transposon 3′ and 5′ ends to mobilize the inserted sequences; a White marker gene; an expression unit containing the TATA region of hsp70 enhancer and the 3′ untranslated region of α-tubulin gene. The expression unit contains a first multiple cloning site (MCS) designed for insertion of an enhancer and a second MCS located 500 bases downstream, designed for the insertion of a gene of interest.

As an alternative to transposable elements, homologous recombination or gene targeting techniques may be used to substitute a gene of interest for one or both copies of the animal's homologous gene. The transgene may be under the regulation of either an exogenous or an endogenous promoter element and may be inserted as either a minigene or a large genomic fragment. In one application, gene function may be analyzed by ectopic expression, using, for example, Drosophila (Brand et al., Methods in Cell Biology (1994) 44:635- 654) or C. elegans (Mello and Fire, Methods in Cell Biology (1995) 48:451-482).

Examples of well-characterized heterologous promoters that may be employed in the creation of transgenic animals include heat shock promoters/enhancers, which are useful for temperature induced mis-expression. In Drosophila, these include the hsp70 and hsp83 genes and in C. elegans, include hsp 16-2 and hsp 16-41. Tissue specific promoters/enhancers may be also useful and in Drosophila, include eyeless (Mozer and Benzer, Development (1994) 120:1049-1058), sevenless (Bowtell et al., PNAS (1991) 88(15):6853-6857) and glass-responsive promoters/enhancers (Quiring et al., Science (1994) 265:785-789) which may be useful for expression in the eye; and enhancers/promoters derived from the dpp or vestigal genes which may be useful for expression in the wing (Staehling-Hampton et al., Cell Growth Differ. (1994) 5(6):585-593; Kim et al., Nature (1996) 382:133-138). Finally, where it may be necessary to restrict the activity of dominant active or dominant negative transgenes to regions in which the pathway is normally active, it may be advantageous to utilize endogenous promoters of genes in the pathway, such as the dmCHT pathway genes.

In C. elegans, examples of preferred tissue specific promoters/enhancers include, but are not limited to, the myo-2 gene promoter (for pharyngeal muscle-specific expression) and the hlh-1 gene promoter (for body-muscle-specific expression). In one embodiment of the present invention, gene fusions for directing the mis-expression of dmCHT pathway genes may be incorporated into a transformation vector which is injected into a nematode along with a plasmid containing a dominant selectable marker, such as rol-6. Transgenic animals may be identified as those exhibiting a roller phenotype and the transgenic animals may be inspected for additional phenotypes of interest created by mis-expression of the dmCHT pathway gene.

In Drosophila, binary control systems employing exogenous DNA may be preferred if testing the mis-expression of genes in a wide variety of developmental stage-specific and tissue-specific patterns. Two examples of binary exogenous regulatory systems include the UAS/GAL4 system from yeast (Hay et al., PNAS (1997) 94(10):5195-5200; Ellis et al., Development (1993) 119(3):855-865) and the “Tet system” derived from E. coli (Bello et al., Development (1998) 125:2193-2202).

The UAS/GAL4 system is a well-established and powerful method of mis-expression in Drosophila which employs the UAS_{G} upstream regulatory sequence for control of promoters by the yeast GAL4 transcriptional activator protein (Brand and Perrimon, Development (1993) 118(2):401-15). In this approach, transgenic Drosophila, termed “target” lines, may be generated wherein the gene of interest to be mis-expressed is operably fused to an appropriate promoter controlled by UAS_{G}. Other transgenic Drosophila strains, termed “driver” lines, may be generated wherein the GAL4 coding region is operably fused to promoters/enhancers that direct the expression of the GAL4 activator protein in specific tissues, such as the eye, wing, nervous system, gut, or musculature. The gene of interest is not expressed in the target lines for lack of a transcriptional activator to drive transcription from the promoter joined to the gene of interest. However, where the UAS-target line is crossed with a GAL4 driver line, mis-expression of the gene of interest may be induced in resulting progeny in a specific pattern characteristic for that GAL4 line. The technical simplicity of this approach makes it possible to sample the effects of directed mis-expression of the gene of interest in a wide variety of tissues by generating one transgenic target line with the gene of interest and crossing that target line with a panel of pre-existing driver lines.

In the “Tet” binary control system, transgenic Drosophila driver lines may be generated wherein the coding region for a tetracycline-controlled transcriptional activator (tTA) is operably fused to promoters/enhancers that direct the expression of tTA in a tissue-specific and/or developmental stage-specific manner. The driver lines may be crossed with transgenic Drosophila target lines wherein the coding region for the gene of interest to be mis-expressed is operably fused to a promoter that possesses a tTA-responsive regulatory element. If the resulting progeny are supplied with food supplemented with a sufficient amount of tetracycline, expression of the gene of interest is blocked. Expression of the gene of interest may be induced simply by removal of tetracycline from the food and the level of expression of the gene of interest may be adjusted by varying the level of tetracycline in the food. The use of the Tet system as a binary control mechanism for mis-expression, therefore, has the advantages of providing a means of controlling the amplitude and timing of mis-expression of the gene of interest, in addition to spatial control. Consequently, if a gene of interest (e.g. a dmCHT gene) has lethal or deleterious effects if mis-expressed at an early stage in development, such as the embryonic or larval stages, the function of the gene of interest in the adult may still be assessed by adding tetracycline to the food during early stages of development and removing tetracycline later so as to induce mis-expression only at the adult stage.

Dominant negative mutations, by which the mutation causes a protein to interfere with the normal function of a wild-type copy of the protein and which may result in loss-of-function or reduced-function phenotypes in the presence of a normal copy of the gene, may be made by known methods (Hershkowitz, Nature (1987) 329:219-222). In the case of active monomeric proteins, overexpression of an inactive form may be achieved, for example, by linking the mutant gene to a highly active promoter and may cause competition for natural substrates or ligands sufficient to significantly reduce net activity of the normal protein. Alternatively, changes to active site residues may be made to create a virtually irreversible association with a target.

B. Assays for Change in Gene Expression

Various expression analysis techniques may be employed in identifying genes differentially expressed between a cell line or an animal expressing a wild type dmCHT gene compared to another cell line or animal expressing a mutant dmCHT gene. Such expression profiling techniques include, but are not limited to, differential display, serial analysis of gene expression (SAGE), transcript profiling coupled to a gene database query, nucleic acid array technology, subtractive hybridization and proteome analysis (e.g. mass-spectrometry and two-dimensional protein gels). Nucleic acid array technology may be employed to determine a global (i.e., genome-wide) gene expression pattern in a normal animal for comparison with an animal having a mutation in dmCHT gene. Gene expression profiling may also be used to identify other genes (or proteins) that may have a functional relation to dmCHT (e.g. may participate in a signaling pathway with the dmCHT gene). The genes may be identified by detecting changes in their expression levels following mutation, i.e., insertion, deletion or substitution in, or over-expression, under-expression, mis-expression or knock-out, of the dmCHT gene.

1. Phenotypes Associated with dmCHT Pathway Gene Mutations

Following isolation of model animals carrying mutated or mis-expressed dmCHT pathway genes or inhibitory RNAs, animals may be carefully examined for phenotypes of interest. For analysis of mutated dmCHT pathway genes (i.e. deletions, insertions and/or point mutations) animal models that are both homozygous and heterozygous for the altered dmCHT pathway gene may be analyzed. Examples of specific phenotypes that may be investigated include, but are not limited to, lethality, sterility, feeding behavior, perturbations in neuromuscular function including alterations in motility and alterations in sensitivity to pesticides and pharmaceuticals.

Some phenotypes more specific to flies include, but are not limited to, alterations in: adult behavior such as, flight ability, walking, grooming, phototaxis, mating or egg-laying; the responses of sensory organs, changes in the morphology, size or number of adult tissues such as, eyes, wings, legs, bristles, antennae, gut, fat body, gonads and musculature; larval tissues such as mouth parts, cuticles, internal tissues or imaginal discs; or larval behavior such as feeding, molting, crawling, or puparian formation; or developmental defects in any germline or embryonic tissues.

Some phenotypes more specific to nematodes include, but are not limited to, locomotory, egg laying, chemosensation, male mating and intestinal expulsion defects. In various cases, single phenotypes or a combination of specific phenotypes in model organisms might point to specific genes or a specific pathway of genes, which facilitate the cloning process.

Genomic sequences containing a dmCHT pathway gene may be employed in confirming whether an existing mutant insect or worm line corresponds to a mutation in one or more dmCHT pathway genes, by rescuing the mutant phenotype. Briefly, a genomic fragment containing the dmCHT pathway gene of interest and potential flanking regulatory regions may be subcloned into any appropriate insect (such as Drosophila) or worm (such as C. elegans) transformation vector and injected into the animals.

For Drosophila, an appropriate helper plasmid is used in the injections to supply transposase for transposon-based vectors. Resulting germline transformants may be crossed for complementation testing to an existing or newly created panel of Drosophila or C. elegans lines whose mutations have been mapped to the vicinity of the gene of interest (Fly Pushing: The Theory and Practice of Drosophila Genetics, supra; and Caenorhabditis elegans: Modern Biological Analysis of an Organism (1995), Epstein and Shakes, eds.). If a mutant line is found to be rescued by this genomic fragment, as judged by complementation of the mutant phenotype, the mutant line likely harbors a mutation in the dmCHT pathway gene. That prediction may be confirmed by sequencing the dmCHT pathway gene from the mutant line to identify the lesion in the dmCHT pathway gene.

2. Identification of Genes that Modify dmCHT Genes

The characterization of new phenotypes created by mutations or misexpression in dmCHT genes may enable testing for genetic interactions between dmCHT genes and other genes that may participate in the same, related, or interacting genetic or biochemical pathway(s). Individual genes may be used as starting points in large-scale genetic modifier screens as described in more detail below. Alternatively, RNAi methods may be used to simulate loss-of-function mutations in the genes being analyzed. It may be of particular interest to investigate whether there are any interactions of dmCHT genes with other well-characterized genes, particularly genes involved in ion transport.

3. Genetic Modifier Screens

A genetic modifier screen using invertebrate model organisms is a particularly preferred method for identifying genes interacting with dmCHT genes, because large numbers of animals may be systematically screened, thereby making it more possible that interacting genes will be identified. In Drosophila, a screen of up to about 10,000 animals may be considered a pilot-scale screen. Moderate-scale screens may employ about 10,000 to about 50,000 flies and large-scale screens may employ greater than about 50,000 flies. In a genetic modifier screen, animals having a mutant phenotype due to a mutation in or misexpression of one or more dmCHT genes may be further mutagenized, for example by chemical mutagenesis or transposon mutagenesis.

The procedures involved in Drosophila genetic modifier screens are well-known in the art (Wolfner and Goldberg, Methods in Cell Biology (1994) 44:33-80; and Karim et al., Genetics (1996) 143:315-329). The procedures differ depending upon the precise nature of the mutant allele being modified. If the mutant allele is genetically recessive, commonly the case in a loss-of-function allele, most males or in some cases females which carry one copy of the mutant allele, may be exposed to an effective mutagen, such as EMS, MMS, ENU, triethylamine, diepoxyalkanes, ICR-170, formaldehyde, X-rays, gamma rays, or ultraviolet radiation. The mutagenized animals may be crossed to animals of the opposite sex also carrying the mutant allele to be modified. Where the mutant allele being modified is genetically dominant, as is commonly the case for ectopically expressed genes, wild type males may be mutagenized and crossed to females carrying the mutant allele to be modified.

The progeny of the mutagenized and crossed flies exhibiting either enhancement or suppression of the original phenotype may be presumed to have mutations in other genes, called “modifier genes”, that participate in the same phenotype-generating pathway. These progeny may be immediately crossed to adults containing balancer chromosomes and used as founders of a stable genetic line. In addition, progeny of the founder adult may be retested under the original screening conditions to ensure stability and reproducibility of the phenotype. Additional secondary screens may be employed, as appropriate, to confirm the suitability of each new modifier mutant line for further analysis.

Standard techniques used for the mapping of modifiers from a genetic screen in Drosophila include, but are not limited to, meiotic mapping with visible or molecular genetic markers, male-specific recombination mapping relative to P-element insertions, complementation analysis with deficiencies, duplications and lethal P-element insertions and cytological analysis of chromosomal aberrations (Fly Pushing: Theory and Practice of Drosophila Genetics, supra; Drosophila: A Laboratory Handbook, supra). Genes corresponding to modifier mutations failing to complement a lethal P-element may be cloned by plasmid rescue of the genomic sequence surrounding that P-element. Alternatively, modifier genes may be mapped by phenotype rescue and positional cloning (Sambrook et al., supra).

Newly identified modifier mutations may be tested directly for interaction with other genes of interest known to be involved or implicated with dmCHT genes by the methods described above. Also, the new modifier mutations may be tested for interactions with genes in other pathways that are not believed to be related to ion transport (e.g. nanos in Drosophila). New modifier mutations exhibiting specific genetic interactions with other genes implicated in ion transport, but not interactions with genes in unrelated pathways, may be of particular interest.

The modifier mutations may also be employed to identify “complementation groups”. Two modifier mutations may be considered to fall within the same complementation group if animals carrying both mutations in trans exhibit essentially the same phenotype as animals that are homozygous for each mutation individually and generally are lethal where in trans to each other (Fly Pushing: The Theory and Practice of Drosophila Genetics, supra). Generally, individual complementation groups defined in this way correspond to individual genes.

Where dmCHT modifier genes have been identified, homologous genes in other species may be isolated using procedures based on cross-hybridization with modifier gene DNA probes, PCR-based strategies with primer sequences derived from the modifier genes and/or computer searches of sequence databases. For therapeutic applications related to the function of dmCHT genes, human and rodent homologs of the modifier genes may be of particular interest.

For pesticide and other agricultural applications, homologs of modifier genes in insects and arachnids may be of particular interest. Insects, arachnids and other organisms of interest include, but are not limited to: Isopoda; Diplopoda; Chilopoda; Symphyla; Thysanura; Collembola; Orthoptera, such as Scistocerca spp; Blattoidea, such as Blattella germanica; Dermaptera; Isoptera; Anoplura; Mallophaga; Thysanoptera; Heteroptera; Homoptera, including Bemisia tabaci and Myzus spp.; Lepidoptera including Plodia interpunctella, Pectinophora gossypiella, Plutella spp., Heliothis spp. and Spodoptera species; Coleoptera such as Leptinotarsa, Diabrotica spp., Anthonomus spp. and Tribolium spp.; Hymenoptera; Diptera, including Anopheles spp.; Siphonaptera, including Ctenocephalides felis; Arachnida; and Acarinan, including Amblyoma americanum; and nematodes, including Meloidogyne spp. and Heterodera glycinii.

Although the above-described Drosophila genetic modifier screens may be quite powerful and sensitive, some genes that interact with dmCHT genes still may be missed in this approach, particularly if there is functional redundancy of those genes. That is because the vast majority of the mutations generated in the standard mutagenesis methods will be loss-of-function mutations, whereas gain-of-function mutations, which may reveal genes with functional redundancy, will be relatively rare.

Another method of genetic screening in Drosophila has been developed that focuses specifically on systematic gain-of-function genetic screens (Rorth et al., Development (1998) 125:1049-1057). This method is based on a modular mis-expression system utilizing components of the GAL4/UAS system (described above) wherein a modified P element, termed an “enhanced P” (EP) element, may be genetically engineered to contain a GAL4-responsive UAS element and promoter. Any other transposons may also be used for this system. The resulting transposon may be utilized to randomly tag genes by insertional mutagenesis (similar to the method of P element mutagenesis described above). Thousands of transgenic Drosophila strains, termed EP lines, may be generated with each line containing a specific UAS-tagged gene. This approach takes advantage of the preference of P elements to insert at the 5′-ends of genes. Consequently, many of the genes tagged by insertion of EP elements become operably fused to a GAL4-regulated promoter and increased expression or mis-expression of the randomly tagged gene may be induced by crossing in a GAL4 driver gene.

Systematic gain-of-function genetic screens for modifiers of phenotypes induced by mutation or mis-expression of a dmCHT gene may be performed by crossing several thousand Drosophila EP lines individually into a genetic background containing a mutant or mis-expressed dmCHT gene and further containing an appropriate GAL4 driver transgene. It may also be possible to remobilize the EP elements to obtain novel insertions. The progeny of these crosses may be analyzed for enhancement or suppression of the original mutant phenotype as described above. Those identified as having mutations which interact with the dmCHT gene may be tested further to verify the reproducibility and specificity of this genetic interaction. EP insertions which demonstrate a specific genetic interaction with a mutant or mis-expressed dmCHT gene, have a physically tagged new gene that may be identified and sequenced using PCR or hybridization screening methods, allowing the isolation of the genomic DNA adjacent to the position of the EP element insertion.

The present invention will now be described for purposes of illustration and not limitation by the following examples.

### EXAMPLES

The details of the conditions used for the identification and/or isolation of the dmCHT nucleic acid and protein of the present invention are described in the Examples section below. Those examples set forth the isolation and cloning of the nucleotide sequence of SEQ ID NO:1 and how the sequence, fragments and derivatives thereof of the present invention, as well as other dmCHT pathway nucleic acids and gene products may be used for genetic studies to elucidate mechanisms of the dmCHT pathway. The examples also describe how the polypeptide of SEQ ID NO:2 and fragments and derivatives thereof may be used in the discovery of potential pharmaceutical or pesticidal agents that interact with the pathway.

### Example 1

### Preparation of Drosophila cDNA Library and Sequencing

A Drosophila expressed sequence tag (EST) cDNA library was prepared as follows. Tissue from mixed stage embryos (0-20 hour), imaginal disks and adult fly heads were collected and total RNA was prepared. Mitochondrial rRNA was removed from the total RNA by hybridization with biotinylated rRNA specific oligonucleotides and the resulting RNA was selected for polyadenylated mRNA. The resulting material was used to construct a random primed library.

First strand cDNA synthesis was primed using a six nucleotide random primer. The first strand cDNA was tailed with terminal transferase to add approximately 15 dGTP molecules. The second strand was primed using a primer that contained a Not1 site followed by a 13 nucleotide C-tail to hybridize to the G-tailed first strand cDNA. The double-stranded cDNA was ligated with BstX1 adaptors and digested with Not1. The cDNA was fractionated according to size by electrophoresis on an agarose gel and the cDNA greater than 700 bp was purified. The CDNA was ligated with Not1, BstX1 digested pCDNA-sk+ vector (a derivative of pBluescript, Stratagene) and used to transform E. coli (XL1 blue). The final complexity of the library was 6×10⁶ independent clones.

The cDNA library was normalized using a modification of the method described by Bonaldo et al. (Genome Research (1996) 6:791-806.). Biotinylated driver was prepared from the cDNA by PCR amplification of the inserts and hybridized with single-stranded plasmids of the same library. The resulting double-stranded forms were removed with strepavidin magnetic beads and the remaining single-stranded plasmids were converted to double-stranded molecules using Sequenase (Amersham, Arlington Hills, Ill.). The plasmid DNA was stored at −20° C. prior to transformation. Aliquots of the normalized plasmid library were used to transform E. coli (XL1 blue or DH10B) plated at moderate density and the colonies picked into a 384-well master plate containing bacterial growth media using a Qbot robot (Genetix, Christchurch, UK). The clones grew for 24 hours at 37° C. and the master plates were frozen at −80° C. for storage. The total number of colonies picked for sequencing from the normalized library was 240,000.

The master plates were used to inoculate media for growth and preparation of DNA for use as template in sequencing reactions. Those reactions were primarily carried out with primer that initiated at the 5′ end of the cDNA inserts. However, a minor percentage of the clones were also sequenced from the 3′ end. Clones were selected for 3′ end sequencing based on either further biological interest or the selection of clones that could extend assemblies of contiguous sequences (“contigs”) as discussed below. DNA sequencing was carried out using ABI377 automated sequencers and used either ABI FS, dirhodamine or BigDye chemistries (Applied Biosystems, Inc., Foster City, Calif.).

### Example 2

### Analysis of dmCHT Nucleotide Sequences

Analyses of sequences were done as follows: the traces generated by the automated sequencers were base-called using the program “Phred” (Gordon, Genome Res. (1998) 8:195-202), which also assigned quality values to each base. The resulting sequences were trimmed for quality in view of the assigned scores. Vector sequences were also removed. Each sequence was compared to all other fly EST sequences using the BLAST program and a filter to identify regions of near 100% identity. Sequences with potential overlap were assembled into “contigs” (an assembly of contiguous sequences) using the programs “Phrap”, “Phred” and “Consed” (Phil Green, University of Washington, Seattle, Wash.; http://bozeman.mbt.washington.edu/phrap.docs/phrap.html).

The resulting assemblies were compared to existing public databases and homology to known proteins was used to direct translation of the consensus sequence. Where no BLAST homology was available, the statistically most likely translation based on codon and hexanucleotide preference was used. The Pfam (Bateman et al., Nucleic Acids Res. (1999) 27:260-262) and Prosite (Hofmann et al., Nucleic Acids Res. (1999) 27(1):215-219) collections of protein domains were used to identify motifs in the resulting translations. The contig sequences were archived in an Oracle-based relational database (FlyTag™, Exelixis, Inc., South San Francisco, Calif.). The dmCHT sequence was predicted from cDNA contigs and available public genomic sequence based on the human and worm homologs.

The dmCHT sequences were analyzed using Pfam and Prosite. The dmCHT of the present invention was a 610 amino acid protein with 12 predicted transmembrane domains. Pfam predicted the dmCHT to be a sodium:solute symporter family member (PF00474).

Nucleotide and amino acid sequences for the dmCHT nucleotide sequence and encoded polypeptide of the present invention were searched against all available nucleotide and amino acid sequences in the public databases, using BLAST (Altschul et al., supra). Table I below lists the most similar sequences.

TABLE I

| | |
|---|---|
| GI Number | DESCRIPTION |

DNA BLAST

| | |
|---|---|
| GBAE003723.3 | Drosophila melanogaster genomic scaffold |
| | 142000013386035 section 105, complete sequence |
| GBAW940114.1 | GH02984.3 prime GH Drosophila melanogaster head |
| | pOT2 cDNA clone GH02984.3 |

PROTEIN BLAST

| | |
|---|---|
| AAF55583.1 | CG gene product (Drosophila melanogaster) |
| AB030946 | High-affinity choline transporter CHO-1 |
| | (Caenorhabditis elegans) |
| AF276871 | High-affinity choline transporter (Homo sapiens) |
| AJ401467 | High-affinity choline transporter (Mus musculus) |
| AB030947 | High-affinity choline transporter CHT1 (Rattus |
| | norvegicus) |

The closest homolog predicted by BLAST analysis was a partial protein prediction (AAF55583.1) from Drosophila, wherein amino acids 1-213 were 100% identical to amino acids 1-213 of SEQ ID NO:2.

The BLAST analysis also revealed several other choline transporter proteins which shared significant amino acid homology (52% identity; 66% similarity) with dmCHT. Taken together, these results suggested that the dmCHT of the present invention functions as choline transporter and thus could be exploited as a target to control disease vectors and insect pests. BLAST results for the dmCHT amino acid sequence indicated 214 amino acid residues as the shortest stretch of contiguous amino acids that differed from other listed sequences and 214 amino acids as the shortest stretch of contiguous amino acids for which there were no sequences contained within public database sharing 100% sequence similarity.

### Example 3

### Testing of Pesticide Compounds for Activity Against Channel Complexes

The cDNAs encoding the dmCHT of the present invention were cloned into mammalian cell culture-compatible vectors (e.g. pCDNA, Invitrogen, Carlsbad, Calif.) and the resultant constructs were transiently transfected into mammalian cells. Those transiently transfected cell lines were used preferably 24 to 48 hours following transfection for electrophysiology studies.

Whole cell recordings, using the voltage clamp technique, were taken on the transfected cells versus cells transfected with vector only. Cells were voltage-clamped at B60 mV and continuously superfused with ND96 (96 mM NaCl, 2 mM KCl, 1.8 mM CaCl₂ 1mM MgCl₂, 5 mM HEPES, pH7.5) containing varying concentrations of compounds. Current and fluxes were measured.

Also, cell lines transiently transfected with dmCHT were assayed for uptake of radioactive or fluorescent choline. For radioactive compounds, cells were incubated in 0.5pM radioactive (³H—, or ¹⁴C—) choline for about one hour, washed with saline and assayed for compound uptake using a scintillation counter. Appropriate controls were comparison of this uptake to uptake in cells injected with water, or noninjected cells.

### Example 4

### Binding Measurements for dmCHT

Equilibrium binding of tritiated compounds with cells expressing the dmCHT of the present invention was measured by a filtration assay. Briefly, 60 nM membrane-bound receptor was incubated with increasing concentrations of tritiated compounds in BC³H1 extracellular buffer (145 mM NaCl/5.3 mM KCl/1.8 mM CaCl₂•2H₂O/1.7 mM MgCl₂•6H₂O/25 mM Hepes, pH 7.4), to give a final volume of 30 μl, for 40 min at 25° C. GF/F glass fiber filters (1.3 cm diameter) (Whatman) were presoaked in 1% Sigmacote in BC³H1 buffer (Sigma) for three hours, aligned in a 96-well Minifold Filtration Apparatus (Schleicher & Schuell) and placed on top of one 11×14 cm GB002 gel blotting paper sheet (Schleicher & Schuell). Thirty-five microliters of each reaction mixture was spotted per well and washed twice with 200 μl ice-cold BC3H1 buffer. The filter-bound radioactivity was quantified by scintillation counting. Saturation curves were constructed by varying the tritiated compound concentration from 50 nM to 10 μM. The amount of nonspecific binding was determined in the presence of non-radioactive analogs of the tritiated compounds.

### Example 5

### Assay of Compounds on Cell Cultures

Compounds that modulate (e.g. block or enhance) dmCHT ion channels were assayed using cultured cells. Cultured mammalian or insect cells (e.g. HEK 293, SF 9) were either transiently or stably transfected with DNA vectors containing the dmCHT gene. Ionic currents passing through the expressed channels were recorded by patch-clamp technique (Hamill et al., Pflugers Arch. (1981) 391(2): 85-100). Solutions containing interesting compounds were screened by passing through the recording cell and monitoring the current or cell membrane potential changes.

### Example 6

### Cell-Based Assay Employing Imaging Techniques

Fluorescent membrane potential dyes were used in monitoring cell membrane potential changes induced by dmCHT activity. Membrane-bound charged fluorescent molecules were added to the cell membrane. As membrane potential changed, the position of the fluorophore was affected. A change of the fluorophore's quenching environment gave a fluorescent signal, which, was used to calibrate the membrane potentials.

Two-component dye systems in which changes in transmembrane potential are detected via fluorescent resonant energy transfer (FRET) between a membrane-bound fluorophore and a charged, membrane-mobile fluorophore have also been developed recently. (Gonzalez et al., Chem Biol. (1997) 4(4):269-77; Cacciatore et al., Neuron (1999) 23:449-59). The sensitivity of this system is governed by electrodiffusion and, in practice, is much higher than that achieved with traditional voltage-sensitive dyes, in which a single chromophore interacts directly with the transmembrane electric field.

The foregoing examples of the present invention are offered for the purpose of illustration and not limitation. It will be apparent to those skilled in the art that the embodiments described herein may be modified or revised in various ways without departing from the spirit and scope of the invention. The scope of the invention is to be measured by the appended claims.

References

1. Alam and Cook, Anal. Biochem. (1990)188:245-254.

2. Allen et al., Trends Biochem. Sci. Trends Biochem. Sci. (1995) 20:511-516.

3. Altschul et al., J. Mol. Biol. (1997) 215:403-410.

4. Apparsundaram S, et al., Biochem Biophys Res Commun 2000 276:862-867.

5. Arriza et al., J. Biol. Chem. (1993) 268:15329-15332.

6. Arriza et al., J. Neurosci.(1994) 14:5559-5569.

7. Ashburner, In Drosophila melanogaster: A Laboratory Manual (1989), Cold Spring Harbor, N.Y., Cold Spring Harbor Laboratory Press: pp. 299-418.

8. Atkinson et al., Proc. Natl. Acad. Sci. U.S.A. (1993) 90:9693-9697.

9. Bailey, Ullmann's Encyclopedia of Industrial Chemistry (1998), 6th ed. Wiley and Sons.

10. Bartel et al., BioTechniques (1993) 14:920-924.

11. Bartel et al., Cellular Interactions in Development (1993) Hartley, ed., Practical Approach Series xviii, IRL Press at Oxford University Press, New York, N.Y., pp. 153-179.

12. Bateman et al., Nucleic Acids Res. (1999) 27:260-262.

13. Bell et al., J. Biol. Chem. (1998) 273:14309-14314).

14. Bellen et al., Genes Dev. (1989) 3(9):1288-1300.

15. Bello et al., Development (1998) 125:2193-2202.

16. Bingham Cell (1997) 90(3):385-387.

17. Bolger, Drug Discovery Today (1999) 4:251-253.

18. Bonaldo et al. Genome Research (1996) 6:791-806.

19. Bowden et al., Science (1998) 280:2129-2132.

20. Bowtell et al., PNAS (1991) 88(15):6853-6857.

21. Brand and Perrimon, Development (1993) 118(2):401-15.

22. Brand et al., Methods in Cell Biology (1994) 44:635- 654.

23. Brenner, Genetics (1974) 110:421-440.

24. Brent and Finley, Annu. Rev. Genet. (1997) 31:663-704.

25. Burns et al., In Vitro Cell Dev Biol Anim (1996) 32:78-84.

26. Burns et al., Proc. Natl. Acad. Sci. USA (1993) 90:8033-37.

27. Cacciatore et al., Neuron (1999) 23:449-59.

28. Caenorhabditis elegans: Modern Biological Analysis of an Organism (1995), Epstein and Shakes, eds.

29. Carter et al., Nucl. Acids Res. (1986) 13:4331.

30. Chalfie et al., Science (1994) 263:802-805.

31. Chasman et al., Mol. Cell. Biol. (1989) 9:4746-4749.

32. Chen et al., Hum. Gen. Ther. (1994) 5:595-601.

33. Chien et al., PNAS (1991) 88:9572-9582.

34. Cole et al., in Monoclonal Antibodies and Cancer Therapy (1985) Alan R. Liss, pp. 77-96.

35. Cole et al., PNAS (1983) 80:2026-2030.

36. Cooley et al., Science (1988) 239:1121-1128.

37. Cory and Bishop, Mol. Biotechnol. (1997) 7(3):303-13.

38. Cumberledge and Krasnow (1994) Methods in Cell Biology 44:143-159. 17. Golic and Lindquist (1989) Cell 59(3):499-509.

39. Current Protocol in Molecular Biology, Vol. 1, Chap. 2.10, John Wiley & Sons, Publishers (1994).

40. Current Protocols in Protein Science (1998) Coligan et al., eds., John Wiley & Sons, Inc., Somerset, N.J.

41. DiCiommo and Bremner, J. Biol. Chem. (1998) 273:18060-66.

42. Dingwall and Laskey, Trends Biochem. Sci. Trends Biochem. Sci. (1991) 16:479-481.

43. Dixon lab, University of Michigan, Clemens et al. PNAS, Jun. 6, 2000, vol. 97, no. 12, pp. 6499-6503.

44. DNA Cloning: A Practical Approach, Vol. 1, 2, 3, 4, (1995) Glover, ed., MRL Press, Ltd., Oxford, U.K.

45. Ellington. et al., Biotechnol. Annu. Rev. (1995) 1:185-214.

46. Ellis et al., Development (1993) 119(3):855-865.

47. Estruch et al., Nat. Biotechnol (1997) 15(2):137-141.

48. Fearon et al., PNAS (1992) 89:7958-7962.

49. Fields and Song, Nature (1989) 340:245-246.

50. Fields and Sternglanz, Trends In Genetics (1994) 10:286-292.

51. Fire et al., Nature (1998) 391:806-811.

52. Fly pushing: The Theory and Practice of Drosophila melanogaster Genetics (1997) Cold Spring Harbor Press, Plainview, N.Y.

53. Fraley et al., Proc. Natl. Acad. Sci. USA (1983) 80:4803.

54. Geisler et al., Cell (1992) 71(4):613-621).

55. Gill et al., Annu Rev Entomol (1992) 37:615-636.

56. Gonzalez et al., Chem Biol. (1997) 4(4):269-77.

57. Good et al., Gene Therapy (1997) 4:45-54.

58. Gordon, Genome Res. (1998) 8:195-202.

59. Gyuris et al., Cell (1993) 75:791-803.

60. Hamill et al., Pflugers Arch. (1981) 391(2): 85-100.

61. Hamilton and Zinn, Methods in Cell Biology (1994) 44:81-94.

62. Hamilton et al., PNAS (1991) 88:2731-2735.

63. Harnessing the power of Drosophila genetics, In Drosophila melanogaster: Practical Uses in Cell and Molecular Biology, Goldstein and Fyrberg, Eds., Academic Press, Inc. San Diego, Calif.

64. Hassanzadeh et al., Febs Lett. (1998) 16(1, 2):75-80 and 81-86.

65. Hay et al., Development (1994) 120:2121-2129.

66. Hay et al., PNAS (1997) 94(10):5195-5200.

67. Hershkowitz, Nature (1987) 329:219-222.

68. Higgs et al., Insect Mol. Biol. (1995) 4:97-103.

69. Hofmann et al., Nucleic Acids Res. (1999) 27(1):215-219.

70. Hohn et al., 1982, In Molecular Biology of Plant Tumors, Academic Press, New York, pp 549-560.

71. Hoogenboom et al., Immunotechnology (1998) 4:1-20.

72. Horsch et al., Science (1984) 233:496-89.

73. http://bozeman.mbt.washington.edu/phrap.docs/phrap.html).

74. http://flybase.bio.indiana.edu.

75. Hunkapiller et al., Nature (1984) 310:105-111.

76. Ivics et al., Cell (1997) 91(4):501-510.

77. Jones et al., Nature (1986) 321:522-525.

78. Jordan et al., Insect Mol Biol (1998) 7: 215-222.

79. Kaiser, Bioassays (1990) 12(6):297-301.

80. Karim et al., Genetics (1996) 143:315-329.

81. Kennerdell and Carthew, Cell (1998) 95:1017-1026.

82. Kim et al., Nature (1996) 382:133-138.

83. Kingston, In Current Protocols in Molecular Biology (1998) Ausubel et al., John Wiley & Sons, Inc. sections 12.0.3-12.10.

84. Klemenz et al., Nucleic Acids Res. (1987) 15(10):3947-3959.

85. Kolonin and Finley, PNAS (1998) 95:14266-14271.

86. Kolonin, M G, and Finley, R L, Genetics, 1998 95:4266-4271.

87. Kricka et al., Molecular Probing, Blotting, and Sequencing (1995) Academic Press, New York, Chapter 1 and Table IX.

88. LaBonne et al., Dev. Biol. (1989) 136(1):1-16.

89. Li and Herskowitz, Science (1993) 262:1870-1874.

90. Luo et al., Biotechniques (1996) 20(4):564-568.

91. Ma et al., Cell (1987) 48:847-853.

92. Margolis and Duyk, Nature Biotech. (1998) 16:311

93. Matsubara et al., Proc. Natl. Acad. Sci. USA (1996) 93:6181-85.

94. Mbungu et al., Archives of Biochemistry and Biophysics (1995) 318:489-497.

95. Mello and Fire, Methods in Cell Biology (1995) 48:451-482.

96. Misquitta and Patterson PNAS (1999) 96:1451-1456.

97. Modern Biological Analysis of an Organism (1995) Epstein and Shakes, Eds.

98. Mozer and Benzer, Development (1994) 120:1049-1058.

99. Nature (1999) 402:370-371.

100. Nucleic Acids Research (1998) 26(1):85-88.

101. O'Brochta et al., Genetics (1996) 142:907-914.

102. O'Kane and Gehring PNAS (1987) 84(24):9123-9127.

103. Okuda T, et al., Nature Neirosci. 2000 3:120-125.

104. Pal-Bhadra et al., Cell (1997) 90(3):479-490.

105. Pasher et al., Gene (1992) 111:229-233.

106. Preston and Engels, Genetics (1996) 144:1611-1638.

107. Ptashne et al., Nature (1990) 346:329-331.

108. Que and Jorgensen, Dev. Genet. (1998) 22(1):100-109.

109. Quiring et al., Science (1994) 265:785-789.

110. Rehm, http://www.fruitfly.org/methods/.

111. Robertson, Insect Physiol. (1995) 41:99-105.

112. Rong and Golic, Science (2000) 288:2013-20018.

113. Rorth et al., Development (1998) 125:1049-1057.

114. Rubin and Spradling, Science (1982) 218(4570):348-353.

115. Sambrook et al., Molecular Cloning, Cold Spring Harbor (1989).

116. Schuh and Jackie, Genome (1989) 31(1):422-425.

117. Seabaugh et al., Virology (1998) 243:99-112.

118. Sheng et al., Genes Devel. (1997) 11:1122-1131.

119. Shi et al., Proc. Natl. Acad. Sci USA (19999) 96:10033-10038.

120. Smyth, Curr. Biol. (1997) 7(12):793-795.

121. Spralding et al., PNAS (1995) 92:0824-10830.

122. Staehling-Hampton et al., Cell Growth Differ. (1994) 5(6):585-593.

123. Steller and Pirrotta, EMBO. J. (1985) 4:167-171.

124. The nematode C. elegans (1988) Wood, Ed., Cold Spring Harbor Laboratory Press, Cold Spring Harbor, N.Y.

125. Thibault et al., Insect Mol Biol (1999) 8(1):119-23.

126. Tower et al. Genetics (1993) 133:347-359.

127. Vasavada et al., PNAS (1991) 88:10686-10690.

128. Vidal et al., PNAS (1996) 93(19):10315-10320.

129. Wada et al., Nucleic Acids Research (1990) 18(Suppl.):2367-241 1.

130. Wells et al., Philos. Trans. R. Soc. London SerA (1986) 317:415.

131. White et al., Science (1996) 271:805-807.

132. Wolfner and Goldberg, Methods in Cell Biology (1994) 44:33-80.

133. Xu et al., PNAS (1997) 94:12473-12478.

134. Ylikomi et al., EMBO J. (1992) 11:3681-3694.

135. Zwaal et al., Proc. Natl. Acad. Sci. U.S.A. (1993) 90:7431-7435.

What is claimed is:

1. An isolated nucleic acid or a complement thereof comprising:
a nucleotide sequence encoding a polypeptide comprising an amino acid sequence having 100% sequence similarity with at least 215 contiguous amino acids of SEQ ID NO:2, or
a nucleotide sequence encoding a functionally active choline transporter comprising an amino acid sequence having at least 80% sequence similarity with SEQ ID NO:2,
wherein said isolated nucleic acid is less than about 15 kb.

2. The isolated nucleic acid of claim 1, wherein said nucleic acid is capable of hybridizing to a nucleic acid comprising SEQ ID NO:1 using hybridization conditions comprising 6× SSC 65° C. and 0.2× SSC wash buffer at 65° C.

3. The isolated nucleic acid of claim 1, wherein said nucleic acid is capable of hybridizing to a nucleic acid comprising SEQ ID NO:1 using hybridization conditions comprising 5× SSC/35% formamide at 40° C. and 2× SSC wash buffer at 55° C.

4. The isolated nucleic acid of claim 1, wherein said nucleic acid is capable of hybridizing to a nucleic acid comprising SEQ ID NO:1 using hybridization conditions comprising 5×SSC/20% formamide at 37° C. and 1×SSC wash buffer at 37° C.

5. The isolated nucleic acid of claim 1, wherein said nucleotide sequence encodes a polypeptide comprising an amino acid sequence having 100% sequence similarity with at least 220 contiguous amino acids of SEQ ID NO:2.

6. The isolated nucleic acid of claim 1, wherein said nucleotide sequence encodes a polypeptide comprising an amino acid sequence having 100% sequence similarity with at least 225 contiguous amino acids of SEQ ID NO:2.

7. The isolated nucleic acid of claim 1, wherein said nucleotide sequence encodes a polypeptide comprising an amino acid sequence having 100% sequence similarity with at least 230 contiguous amino acids of SEQ ID NO:2.

8. The isolated nucleic acid of claim 1, wherein said nucleotide sequence encodes a polypeptide comprising the entire sequence of SEQ ID NO:2.

9. A polypeptide comprising an amino acid sequence having 100% sequence similarity with at least 215 contiguous amino acids of SEQ ID NO:2.

10. A polypeptide comprising an amino acid sequence having at least 80% sequence similarity with SEQ IS NO:2.

11. A polypeptide comprising the amino acid sequence of SEQ ID NO:2.

12. A vector comprising an isolated nucleic acid or a complement thereof including a nucleotide sequence encoding a polypeptide comprising an amino acid sequence having 100% sequence similarity with at least 215 contiguous amino acids of SEQ ID NO:2, or a nucleotide sequence encoding a functionally active choline transporter comprising an amino acid sequence having at least 80% sequence similarity with SEQ ID NO:2,
wherein said isolated nucleic acid is less than about 15 kb.

13. A host cell comprising a vector having an isolated nucleic acid or a complement thereof including a nucleotide sequence encoding a polypeptide comprising an amino acid sequence having 100% sequence similarity with at least 215 contiguous amino acids of SEQ ID NO:2, or a nucleotide sequence encoding a functionally active choline transporter comprising an amino acid sequence having at least 80% sequence similarity with SEQ ID NO:2,
wherein said isolated nucleic acid is less than about 15 kb.

14. A method of producing a Drosophila melanogaster choline transporter (“dmCHT”) comprising:
culturing a host cell comprising a vector comprising an isolated nucleic acid or a complement thereof comprising a nucleotide sequence encoding a polypeptide comprising an amino acid sequence having 100% sequence similarity with at least 215 contiguous amino acids of SEQ ID NO:2, or a nucleotide sequence encoding a functionally active choline transporter comprising an amino acid sequence having at least 80% sequence similarity with SEQ ID NO:2, wherein said isolated nucleic acid is less than about 15 kb; and
recovering said dmCHT.

15. A method of detecting at least one compound which interacts with a Drosophila melanogaster choline transporter (“dmCHT”) or fragment thereof, said method comprising:
exposing a polypeptide comprising an amino acid sequence having 100% sequence similarity with at least 215 contiguous amino acids of SEQ ID NO:2 or a sequence having at least 80% sequence similarity with SEQ ID NO:2 or a fragment thereof to at least one compound; and
detecting at least one interaction between said at least one compound and said polypeptide.

16. The method of claim 15, wherein said at least one compound comprises a putative pesticide or pharmaceutical agent.

17. The method of claim 15, wherein exposing comprises administering said at least one compound to cultured host cells genetically altered to express said dmCHT or fragment thereof.

18. The method of claim 15, wherein exposing comprises administering said at least one compound to an animal genetically altered to express said dmCHT or fragment thereof.

19. The method of claim 18, wherein administering comprises at least one of bathing, feeding, injecting and contacting.

20. The method of claim 18, wherein said compound is a putative pesticide and wherein detecting comprises observing modulations of dmCHT activity resulting in animal lethality.

21. The method of claim 18, wherein said animal is selected from insects, arachnids and worms.

22. The method of claim 18, wherein said animal is selected from Isopoda, Diplopoda, Chilopoda, Symphyla, Thysanura, Collembola, Orthoptera, Blattoidea, Dermaptera, Isoptera, Anoplura, Mallophaga, Thysanoptera, Heteroptera, Homoptera, Lepidoptera, Coleoptera, Spodoptera, Hymenoptera, Diptera, Siphonaptera, Arachnida, Acarinan and nematodes.

23. The method of claim 22, wherein said animal is selected from Scistocerca spp., Blattella germanica, Bemisia tabaci, Myzus spp., Plodia interpunctella, Pectinophora gossypiella, Plutella spp., Heliothis spp., Leptinotarsa, Diabrotica spp., Anthonomus spp., Tribolium spp., Anopheles spp., Ctenocephalides felis, Amblyoma americanum, Meloidogyne spp. and Heterodera glycinii.

24. An animal genetically altered to express or mis-express a Drosophila melanogaster choline transporter (“dmCHT”), or the progeny thereof which has inherited that dmCHT expression or mis-expression mutation,
wherein said dmCHT comprises an amino acid sequence having 100% sequence similarity with at least 215 contiguous amino acids of SEQ ID NO:2 or a sequence having at least 80% sequence similarity with SEQ ID NO:2.

25. The animal of claim 24, selected from insects, arachnids and worms.

26. The animal of claim 24, selected from Isopoda, Diplopoda, Chilopoda, Symphyla, Thysanura, Collembola, Orthoptera, Blattoidea, Dermaptera, Isoptera, Anoplura, Mallophaga, Thysanoptera, Heteroptera, Homoptera, Lepidoptera, Coleoptera, Spodoptera, Hymenoptera, Diptera, Siphonaptera, Arachnida, Acarinan and nematodes.

27. The animal of claim 26, selected from Scistocerca spp., Blattella germanica, Bemisia tabaci, Myzus spp., Plodia interpunctella, Pectinophora gossypiella, Plutella spp., Heliothis spp., Leptinotarsa, Diabrotica spp., Anthonomus spp., Tribolium spp., Anopheles spp., Ctenocephalides felis, Amblyoma americanum, Meloidogyne spp. and Heterodera glycinii.

28. A method of studying Drosophila melanogaster choline transporter (“dmCHT”) activity comprising:
detecting the phenotype caused by expression or mis-expression of dmCHT in an animal genetically altered to express or mis-express said dmCHT and/or the progeny thereof which has inherited that dmCHT expression or mis-expression mutation,
wherein said dmCHT comprises an amino acid sequence having 100% sequence similarity with at least 215 contiguous amino acids of SEQ ID NO:2 or a sequence having at least 80% sequence similarity with SEQ ID NO:2.

29. The method of claim 28 further including administering at least one compound to said animal and/or said progeny thereof and observing changes in dmCHT activity of said animal and/or said progeny thereof.

30. The method of claim 28, wherein said animal is selected from insects, arachnids and worms.

31. The method of claim 28, wherein said animal is selected from Isopoda, Diplopoda, Chilopoda, Symphyla, Thysanura, Collembola, Orthoptera, Blattoidea, Dermaptera, Isoptera, Anoplura, Mallophaga, Thysanoptera, Heteroptera, Homoptera, Lepidoptera, Coleoptera, Spodoptera, Hymenoptera, Diptera, Siphonaptera, Arachnida, Acarinan and nematodes.

32. The method of claim 31, wherein said animal is selected from Scistocerca spp., Blattella germanica, Bemisia tabaci, Myzus spp., Plodia interpunctella, Pectinophora gossypiella, Plutella spp., Heliothis spp., Leptinotarsa, Diabrotica spp., Anthonomus spp., Tribolium spp., Anopheles spp., Ctenocephalides felis, Amblyoma americanum, Meloidogyne spp. and Heterodera glycinii.

33. The method of claim 28, wherein said phenotype is selected from lethality, sterility, feeding behavior, perturbations in neuromuscular function, alterations in motility, alterations in sensitivity to pesticides and pharmaceuticals, flight ability, walking, grooming, phototaxis, mating, egg-laying, responses of sensory organs, changes in the morphology, size or number of eyes, wings, legs, bristles, antennae, gut, fat body, gonads and musculature, mouth parts, cuticles, internal tissues, imaginal discs, molting, crawling, puparian formation and developmental defects in germline or embryonic tissues.

34. A method of studying Drosophila melanogaster choline transporter (“dmCHT”) activity comprising:
detecting the phenotype caused by expression or mis-expression of a dmCHT in a first animal genetically altered to express or mis-express said dmCHT, and/or the progeny thereof which has inherited that dmCHT expression or mis-expression mutation, wherein said dmCHT comprises an amino acid sequence 100% sequence similarity with at least 215 contiguous amino acids of SEQ ID NO:2 or a sequence having at least 80% sequence similarity with SEQ ID NO:2;
detecting the phenotype of a second animal having the same genetic mutation as said first animal and a mutation in a gene of interest; and
observing at least one difference between the phenotype of said first animal and the phenotype of said second animal,
wherein the said at least one difference identifies said gene of interest as capable of modifying the function of the gene encoding said dmCHT.

35. The method of claim 34, wherein said first and said second animals are selected from insects, arachnids and worms.

36. The method of claim 34, wherein said first and said second animals are selected from Isopoda, Diplopoda, Chilopoda, Symphyla, Thysanura, Collembola, Orthoptera, Blattoidea, Dermaptera, Isoptera, Anoplura, Mallophaga, Thysanoptera, Heteroptera, Homoptera, Lepidoptera, Coleoptera, Spodoptera, Hymenoptera, Diptera, Siphonaptera, Arachnida, Acarinan and nematodes.

37. The method of claim 35, wherein said first and said second animals are selected from Scistocerca spp., Blattella germanica, Bemisia tabaci, Myzus spp., Plodia interpunctella, Pectinophora gossypiella, Plutella spp., Heliothis spp., Leptinotarsa, Diabrotica spp., Anthonomus spp., Tribolium spp., Anopheles spp., Ctenocephalides felis, Amblyoma americanum, Meloidogyne spp. and Heterodera glycinii.

38. The method of claim 34, wherein said phenotype is selected from lethality, sterility, feeding behavior, perturbations in neuromuscular function, alterations in motility, alterations in sensitivity to pesticides and pharmaceuticals, flight ability, walking, grooming, phototaxis, mating, egg-laying, responses of sensory organs, changes in the morphology, size or number of eyes, wings, legs, bristles, antennae, gut, fat body, gonads and musculature, mouth parts, cuticles, internal tissues, imaginal discs, molting, crawling, puparian formation and developmental defects in germline or embryonic tissues.

39. A biopesticide comprising:
an isolated nucleic acid or a complement thereof having a nucleotide sequence encoding a polypeptide comprising an amino acid sequence having 100% sequence similarity with 215 contiguous amino acids of SEQ ID NO:2, or
a nucleotide sequence encoding a functionally active choline transporter comprising an amino acid sequence having at least 80% sequence similarity with SEQ ID NO:2, or
a vector comprising an isolated nucleic acid or a complement thereof having a nucleotide sequence encoding a polypeptide comprising an amino acid sequence having 100% sequence similarity with at least 215 contiguous amino acids of SEQ ID NO:2, or a nucleotide sequence encoding a functionally active choline transporter comprising an amino acid sequence having at least 80% sequence similarity with SEQ ID NO:2,
wherein said isolated nucleic acid is less than about 15 kb.

40. The biopesticide of claim 39, wherein said nucleotide sequence encodes a polypeptide comprising an amino acid sequence having 100% sequence similarity with at least 220 contiguous amino acids of SEQ ID NO:2.

41. The biopesticide of claim 39, wherein said nucleotide sequence encodes a polypeptide comprising an amino acid sequence having 100% sequence similarity with at least 225 contiguous amino acids of SEQ ID NO:2.

42. The biopesticide of claim 39, wherein said nucleotide sequence encodes a polypeptide comprising an amino acid sequence having 100% sequence similarity with at least 230 contiguous amino acids of SEQ ID NO:2.

43. The biopesticide of claim 39, wherein said nucleotide sequence encodes a polypeptide comprising SEQ ID NO:2.

44. The biopesticide of claim 39 further including at least one of a carrier and a surfactant.

45. A method of controlling the growth of at least one pest, said method comprising applying to said pest and/or its locus an effective amount of a biopesticide comprising an isolated nucleic acid or a complement thereof having a nucleotide sequence encoding a polypeptide comprising an amino acid sequence having 100% sequence similarity with 215 contiguous amino acids of SEQ ID NO:2, or a nucleotide sequence encoding a functionally active choline transporter comprising an amino acid sequence having at least 80% sequence similarity with SEQ ID NO:2, or a vector comprising an isolated nucleic acid or a complement thereof having a nucleotide sequence encoding a polypeptide comprising an amino acid sequence having 100% sequence similarity with at least 215 contiguous amino acids of SEQ ID NO:2, or a nucleotide sequence encoding a functionally active choline transporter comprising an amino acid sequence having at least 80% sequence similarity with SEQ ID NO:2, wherein said isolated nucleic acid is less than about 15 kb.

46. The method of claim 45, wherein said at least one pest is selected from insects, arachnids and worms.

47. The method of claim 45, wherein said at least one pest is selected from Isopoda, Diplopoda, Chilopoda, Symphyla, Thysanura, Collembola, Orthoptera, Blattoidea, Dermaptera, Isoptera, Anoplura, Mallophaga, Thysanoptera, Heteroptera, Homoptera, Lepidoptera, Coleoptera, Spodoptera, Hymenoptera, Diptera, Siphonaptera, Arachnida, Acarinan and nematodes.

48. The method of claim 45, wherein said at least one pest is selected from Scistocerca spp., Blattella germanica, Bemisia tabaci, Myzus spp., Plodia interpunctella, Pectinophora gossypiella, Plutella spp., Heliothis spp., Leptinotarsa, Diabrotica spp., Anthonomus spp., Tribolium spp., Anopheles spp., Ctenocephalides felis, Amblyoma americanum, Meloidogyne spp. and Heterodera glycinii.

49. An antibody which specifically binds to a polypeptide comprising an amino acid sequence having 100% sequence similarity with at least 215 contiguous amino acids of SEQ ID NO:2.

50 An antibody with specifically binds to a polypeptide comprising an amino acid sequence having 100% sequence similarity with SEQ ID NO:2.

51. A method of controlling the growth of at least one pest, said method comprising applying to said pest and/or its locus an effective amount of a biopesticide comprising an isolated nucleic acid or a complement thereof having a nucleotide sequence encoding a polypeptide comprising an amino acid sequence of SEQ ID NO:2, or a vector comprising an isolated nucleic acid or a complement thereof having a nucleotide sequence encoding a polypeptide comprising an amino acid sequence of SEQ ID NO:2, wherein said isolated nucleic acid is less than about 15 kb.

52. The method of claim 51, wherein said at least one pest is selected from insects, arachnids and worms.

53. The method of claim 51, wherein said at least one pest is selected from Isopoda, Diplopoda, Chilopoda, Symphyla, Thysanura, Collembola, Orthoptera, Blattoidea, Dermaptera, Isoptera, Anoplura, Mallophaga, Thysanoptera, Heteroptera, Homoptera, Lepidoptera, Coleoptera, Spodoptera, Hymenoptera, Diptera, Siphonaptera, Arachnida, Acarinan and nematodes.

54. The method of claim 51, wherein said at least one pest is selected from Scistocerca spp., Blattella germanica, Bemisia tabaci, Myzus spp., Plodia interpunctella, Pectinophora gossypiella, Plutella spp., Heliothis spp., Leptinotarsa, Diabrotica spp., Anthonomus spp., Tribolium spp., Anopheles spp., Ctenocephalides felis, Amblyoma americanum, Meloidogyne spp. and Heterodera glycinii.
